(19) United States
(12) Patent Application Publication
(10) Pub. No.: US 2024/0058363 A1
(43) Pub. Date: Feb. 22, 2024

(54) USE OF CANNABIDIOL FOR THE TREATMENT OF PSYCHOLOGICAL DISTRESS

(71) Applicant: Emyria, Leederville (AU)

(72) Inventors: Sebastian Roth, Crawley (AU); Alistair Vickery, Floreat (AU)

(73) Assignee: Emyria, Leederville (AU)

(21) Appl. No.: 18/035,396

(22) PCT Filed: Oct. 22, 2021
(86) PCT No.: PCT/AU2021/051235

§ 371 Date: May 4, 2023

(30) Foreign Application Priority Data
Nov. 12, 2020 (AU) 2020904152
Apr. 14, 2021 (AU) 2021901086

(51) Int. Cl. A61K 31/00; A61P 25/22; A61P 25/24; A61P 25/20

(52) U.S. Cl. CPC A61K 31/658; A61P 25/22; A61P 25/24; A61P 25/20

(57) ABSTRACT

A method of treating or preventing one or more symptoms of psychological distress in a subject comprising the step of: a) administering a dose of between 50 mg/day and 200 mg/day cannabidiol for at least one week; wherein the subject has symptoms of at least one of anxiety, stress and/or depression when assessed by a validated scale or biomarker for identifying symptoms of anxiety, stress and/or depression.

### TECHNICAL FIELD

The present invention relates to method and composition comprising cannabidiol for the treatment or prevention of symptoms of psychological distress such as anxiety, stress and/or depression and insomnia.

### BACKGROUND ART

Anxiety, depression and stress are symptoms of psychological distress and are the most prevalent psychiatric symptoms, being associated with a high burden of illness. For example, up to one-third of women and one-fifth of men will experience anxiety at some point in their lives. Insomnia is a further significant health burden, often associated with psychological distress.

There are a number of psychological and medical treatments available to alleviate anxiety, stress and depression and insomnia.

Psychological treatments include cognitive behavioural therapy (CBT), counselling and behavioural therapy. These can be effective, but are time intensive and costly, and not easily accessed for some subjects, particularly in rural areas.

Medical treatments include the administration of antidepressant medications (including SSRIs and SNRIs), beta blockers and benzodiazepines. However, many of these medications can have significant side effects such as nausea, drowsiness, weight gain and addiction.

The present invention seeks to provide a composition and method to reduce or prevent symptoms related to anxiety stress and depression and insomnia, or to provide the consumer with a useful or commercial choice.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present method provides a method of treating or preventing one or more symptoms of psychological distress in a subject comprising the step of:
a) administering a dose of between 50 mg/day and 200 mg/day cannabidiol for at least one week
   wherein the subject has symptoms of at least one of anxiety, stress and/or depression when assessed by a validated scale or biomarker for identifying symptoms of anxiety, stress and/or depression.

The present method provides a method of treating or preventing one or more symptoms of psychological distress in a subject comprising the step of:
a) administering a dose of between 50 mg/day and 200 mg/day cannabidiol for at least one week
   wherein the subject has symptoms of at least one of anxiety, stress and/or depression when assessed by a validated scale for identifying symptoms of anxiety, stress and/or depression.

The present method further provides a method of treating or preventing one or more symptoms of psychological distress in a subject comprising the step of:
a) administering a dose of between 50 mg/day and 200 mg/day cannabidiol for at least one week
   wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia.

The present method further provides a method of treating or preventing one or more symptoms of psychological distress in a subject comprising the step of:
a) administering a dose of between 50 mg/day and 200 mg/day cannabidiol for at least one week
   wherein the subject has symptoms of at least one of anxiety, stress and/or depression when assessed by a validated scale for identifying symptoms of anxiety, stress and/or depression and the subject has insomnia as assessed by a validated scale for identifying symptoms of insomnia.

Preferably, the subject has symptoms of two or more of anxiety, stress and/or depression. The subject may alternatively have symptoms of two or more of anxiety, stress and/or depression and symptoms of insomnia.

Preferably the method reduces the subject's score on the validated scale for anxiety or reduces the anxiety biomarker and/or reduces the subject's score on the validated scale for stress or reduces the stress biomarker and/or reduces the subject's score on the validated scale for depression or the depression biomarker. Preferably the method reduces the subject's score on the validated scale for anxiety and/or reduces the subject's score on the validated scale for stress and/or reduces the subject's score on the validated scale for depression. Preferably the method reduces the subject's score on the validated scale for insomnia or reduces the insomnia biomarker.

In one aspect of the invention, the subject does not have a current diagnosis of a mental disorder corresponding to the DSM-5 criteria for: depressive disorders, trauma- and stressor-related disorders, obsessive-compulsive and related disorders. In one aspect of the invention, the subject does not have a current diagnosis of a mental disorder corresponding to the DSM-5 criteria for anxiety disorder.

Preferably the subject has symptoms of anxiety, stress and/or depression or insomnia secondary to a medical condition, more preferably a chronic medical condition. The medical condition may be one of the following: cancer, cancer treatment, chronic diseases such as heart and lung disease or diabetes, migraine, insomnia or sleep disorder, chronic pain; gastrointestinal disorder; neurological disorder, osteoarthritis or inflammatory arthritis. The medical condition may be mental illness that is not a mental disorder corresponding to the DSM-5 criteria for: depressive disorders, anxiety disorder trauma- and stressor-related disorders, obsessive-compulsive and related disorders.

The present invention further provides a composition comprising between 50 mg and 200 mg cannabidiol for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and wherein the total daily dose of cannabidiol is between 50 mg/day and 200 mg/day.

There is further provided a composition comprising between 50 mg and 200 mg cannabidiol for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia and wherein the total daily dose of cannabidiol is between 50 mg/day and 200 mg/day.

The composition may be administered orally, by nasal or pulmonary administration, or administered via the oral mucosa. The composition is preferably administered for at least one week.

According to another aspect of the invention, there is provided the use of a composition comprising between 50 mg and 200 mg cannabidiol for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and wherein the total daily dose of cannabidiol is between 50 mg/day and 200 mg/day and wherein the composition is administered for at least one week.

According to another aspect of the invention, there is provided the use of between 50 mg and 200 mg cannabidiol, in the manufacture of composition for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and wherein the composition is administered for at least one week.

According to another aspect of the invention, there is provided the use of a composition comprising between 50 mg and 200 mg cannabidiol for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia and wherein the total daily dose of cannabidiol is between 50 mg/day and 200 mg/day and wherein the composition is administered for at least one week.

According to another aspect of the invention, there is provided the use of between 50 mg and 200 mg cannabidiol, in the manufacture of composition for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia and wherein the total daily dose of cannabidiol is between 50 mg/day and 200 mg/day and wherein the composition is administered for at least one week.

According to another aspect of the invention, there is provided a kit for the treatment or prevention of one or more symptoms of psychological distress in a subject comprising:
a) a composition comprising between 50 mg and 200 mg cannabidiol;
b) instructions for use
   wherein the subject has symptoms of at least one of anxiety, stress and/or depression when assessed by a validated scale or biomarker for identifying symptoms of anxiety, stress and/or depression and wherein the total daily dose of cannabidiol is between 50 mg/day and 200 mg/day and wherein the composition is administered for at least one week.

There is further provided a kit for the treatment or prevention of one or more symptoms of psychological distress in a subject comprising:
a) a composition comprising between 50 mg and 200 mg of cannabidiol;
b) instructions for use
   wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia and wherein the total daily dose of cannabidiol is between 50 mg/day and 200 mg/day and wherein the composition is administered for at least one week.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a DASS-21 questionnaire and assessment criteria.

FIG. 2 provides a Schedule of Assessments. a: Review Visits are at Doctor's discretion. Suggested one/month for first 6 months but can be more or less. b: May occur at any time after the patient has ceased treatment. c: Optional visits as clinically indicated. d: Obtain if possible. e: As applicable.

FIG. 3 is a graph of mean anxiety scores across DASS-21 categories and by visit (initial and first follow-up using cannabidiol only). A reduction in scores at the first follow-up shows improvement in anxiety. Mean days of treatment is 47 (+/−23). Error bars display the respective 95% confidence intervals. * p<0.05, ** p<0.01, *** p<0.001.

FIG. 4 is a graph of mean stress scores across DASS-21 categories and by visit (initial and first follow-up using cannabidiol only). A reduction in scores at the first follow-up shows improvement in stress. Mean days of treatment is 47 (+/−23). Error bars display the respective 95% confidence intervals. * p<0.05, ** p<0.01, *** p<0.001.

FIG. 5 is a graph of mean depression scores across DASS-21 categories and by visit (initial and first follow-up using cannabidiol only). A reduction in scores at the first follow-up shows improvement in depression. Mean days of treatment is 47 (+/−23). Error bars display the respective 95% confidence intervals. * p<0.05, ** p<0.01, *** p<0.001.

FIG. 6 is a graph of the change in anxiety scores from initial visit to first follow-up and cannabidiol dosage. Lower values of change indicate less anxiety. The solid fitted line represents a LOESS curve with 95% confidence intervals.

FIG. 7 is a graph of the change in stress scores from initial visit to first follow-up and cannabidiol dosage. Lower values of change indicate less stress. The solid fitted line represents a LOESS curve with 95% confidence intervals.

FIG. 8 is a graph of the change in depression scores from initial visit to first follow-up and cannabidiol dosage. Lower values of change indicate less depression. The solid fitted line represents a LOESS curve with 95% confidence intervals.

FIG. 9 is the ISI questionnaire and assessment criteria.

FIG. 10 is a graph of the change in ISI insomnia scores from initial visit to first follow-up and cannabidiol dosage.

FIG. 11 is a graph of Mean ISI scores across baseline ISI categories and by visit (initial and first follow-up) for patients starting with a (i) DASS-21 anxiety, depression, or stress score above the normal range (Panel A); (ii) DASS-21 anxiety score above the normal range (Panel B); (iii) and DASS-21 anxiety score in either the mild or moderate range (Panel C).

FIG. 12 is a graph of CBD dosage across ISI categories.

FIG. 13 is a graph of mean ISI scores over the first six months.

FIG. 14 is a graph of mean DASS-21 anxiety score over the first six months.

FIG. 15 is a graph of mean DASS-21 depression score over the first six months.

FIG. 16 is a graph of mean DASS-21 stress score over the first six months.

### DESCRIPTION OF INVENTION

### Detailed Description of the Invention

### Method of Treatment

The present invention has found that the administration of cannabidiol can assist with treating or preventing one or more symptoms of psychological distress in a subject. The symptoms of psychological distress may be anxiety, stress and/or depression. Additionally, the symptoms may be insomnia. Insomnia is associated with psychological distress and the symptoms of anxiety, stress and/or depression may lead to symptoms of insomnia and equally the symptoms of insomnia may lead to symptoms of anxiety, stress and/or depression.

The present method provides a method of treating or preventing one or more symptoms of psychological distress in a subject comprising the step of:
a) administering a dose of between 50 mg/day and 200 mg/day cannabidiol for at least one week
   wherein the subject has symptoms of at least one of anxiety, stress and/or depression when assessed by a validated scale or biomarker for identifying symptoms of anxiety, stress and/or depression. Preferably, the subject has symptoms of two or more of anxiety, stress and/or depression.

The symptoms of psychological distress may present as insomnia. Preferably, the method of treating or preventing one or more symptoms of psychological distress results in a reduction in insomnia.

Insomnia may present as disturbed sleep, inability to get to sleep, inability to stay asleep and/or sleep that leaves the subject tired upon waking. The insomnia may be assessed using the Insomnia Severity Index (ISI) 7-item self-report questionnaire. Notably, the ISI has been clinically validated to identify and isolate aspects of insomnia severity based on several indicators, such as difficulty falling asleep, staying asleep, waking up too early, degree of impairment with daytime functioning or satisfaction with sleep. More importantly perhaps, this global measure has been employed in number of recent clinical trials to assess the responses in insomnia to a wide variety of treatments.

The present method therefore provides a method of treating or preventing one or more symptoms of psychological distress in a subject comprising the step of:
a) administering a dose of between 50 mg/day and 200 mg/day cannabidiol for at least one week
   wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia. Preferably, the subject has symptoms of two or more of anxiety, stress and/or depression and symptoms of insomnia.

The present method further provides a method of treating or preventing one or more symptoms of psychological distress in a subject comprising the step of:
b) administering a dose of between 50 mg/day and 200 mg/day cannabidiol for at least one week
   wherein the subject has symptoms of at least one of anxiety, stress and/or depression when assessed by a validated scale for identifying symptoms of anxiety, stress and/or depression.

The present method further provides a method of treating or preventing one or more symptoms of psychological distress in a subject comprising the step of:
a) administering a dose of between 50 mg/day and 200 mg/day cannabidiol for at least one week
   wherein the subject has symptoms of at least one of anxiety, stress and/or depression when assessed by a validated scale for identifying symptoms of anxiety, stress and/or depression and the subject has insomnia as assessed by a validated scale or biomarker for identifying symptoms of insomnia.

Preferably, the dose is administered in a single composition once per day. Alternatively, the dose may be delivered two, three or four times in separate compositions, to a total daily dose of between 50 mg and 200 mg cannabidiol.

Preferably the validated scale for identifying symptoms of anxiety, stress and/or depression is chosen from the list comprising: Depression Anxiety Stress Scales (DASS-21, DASS-42), Penn State Worry Questionnaire, Kessler's psychological distress scale 10 (K-10), Hospital Anxiety and Depression Scale (HAD), Hospital Anxiety and Depression Scale-Anxiety Subscale (HADS-A), Beck Anxiety Inventory (BAI), Mood and Anxiety Symptom Questionnaire (MASQ), Generalized Anxiety Disorder Scale (GAD-7), Four-Dimensional Symptom Questionnaire (4DSQ), State-Trait Anxiety Inventory (STAI), Liebowitz Social Anxiety Scale (LSAS), Overall Anxiety Severity and Impairment Scale (OASIS), Patient Health Questionnaire-4 (PHQ-4), Social Interaction Anxiety Scale (SIAS), Social Phobia Inventory (SPIN), Social Phobia and Anxiety Inventory (SPAI), Brief Fear of Negative Evaluation Scale (BFNE), Zung Self-Rating Anxiety/Depression Scale (SAS/SDS), Westside Test Anxiety Scale Validation, Revised Children's Anxiety and Depression Scale (ROADS), Spence Children's Anxiety Scale (SCAS), Anxiety Likert or Visual Analog Scale (VAS), Hamilton Anxiety Rating Scale (HAM-A). In one form, the validated scale is DASS (DASS-21 or DASS-42). Preferably, the validated scale is not HAM-A.

Preferably the validated scale can be administered and scored by non-psychologists. Preferably the validated scale is a self-reported assessment used to isolate and identify aspects of emotional disturbance; for example, to assess the degree of severity of the core symptoms of depression, anxiety or stress. For example, the scale may be DASS (DASS-21 or DASS-42), or K-10.

In one aspect, the validated scale is not a psychological questionnaire used by qualified psychologists to rate the severity of a patient's anxiety. Preferably the validated scale is not a clinical rating of the extensiveness of anxiety intended for individuals that have already been diagnosed with an anxiety disorder or anxiety neurosis. Preferably, the validated scale is not HAM-A.

Preferably the validated scale for identifying symptoms of insomnia is the Insomnia Severity Index (ISI) 7-item self-report questionnaire. Alternative rating sales such as the, Pittsburgh Sleep Quality Index (PSQI), Medical Outcomes Study (MOS) Sleep Scale, Regensburg Insomnia Scale (RIS), Bergen Insomnia Scale (BIS), Athens Insomnia Scale (AIS), sleep diary or sleep apps may be used. The method for identifying symptoms of insomnia may be actigraphy and/or polysomnography (PSG). Preferably the validated scale can be administered and scored by non-psychologists. Preferably the validated scale is a self-reported assessment used to isolate and identify aspects of insomnia and sleep disturbance.

Preferably the method reduces the subject's score on the validated scale for anxiety or reduces the anxiety biomarker. Preferably the method also reduces the subject's score on the validated scale for stress and/or depression or reduces the stress and/or depression biomarker(s). Preferably the method reduces the subject's score on the validated scale for anxiety and/or reduces the subject's score on the validated scale for stress and/or reduces the subject's score on the validated scale for depression.

Preferably the method reduces the subject's score on the validated scale for insomnia or reduces the insomnia biomarker.

The “symptoms of anxiety” include excessive anxiety and worry (apprehensive expectation) about events or activities (such as social events; work or school performance). The anxiety may be associated with restlessness or feeling keyed up or on edge; being easily fatigued; difficulty concentrating or mind going blank; irritability; muscle tension and/or sleep disturbance (difficulty falling or staying asleep; or restless unsatisfying sleep). The term preferably covers undiagnosed or common anxiety, that is anxiety not diagnosed as an anxiety disorder by a qualified health care provider or physician. However, the term may also include diagnosed disorders such as generalized anxiety disorder, agoraphobia, social anxiety disorder, separation anxiety disorder, selective mutism, specific phobia, panic disorder, agoraphobia, substance/medication-induced anxiety disorder, and anxiety disorder due to another medical condition. The anxiety may be an anxiety disorder as defined in the Diagnostic and Statistical Manual of Mental Disorders (DSM-5).

The “symptoms of depression” include feeling sad or having a depressed mood; loss of interest or pleasure in activities once enjoyed; changes in appetite; weight loss or gain unrelated to dieting; trouble sleeping or sleeping too much; loss of energy or increased fatigue; increase in purposeless physical activity (e.g., inability to sit still, pacing, handwringing) or slowed movements or speech; feeling worthless or guilty; difficulty thinking, concentrating or making decisions; thoughts of death or suicide. The term preferably covers undiagnosed depression. However, the term may also include depression diagnosed by a qualified health care provider or physician. The depression may be Major Depressive Disorder as defined in the Diagnostic and Statistical Manual of Mental Disorders (DSM-5).

The “symptoms of stress” include feeling overwhelmed; inability to concentrate; poor judgment; seeing only the negative; anxious or racing thoughts; constant worrying; agitation; moodiness, irritability, or anger; feeling overwhelmed; loneliness and isolation; aches and pains; diarrhea or constipation; nausea, dizziness; chest pain, rapid heart rate; loss of sex drive; frequent colds or flu; eating more or less; sleeping too much or too little; withdrawing from others; procrastinating or neglecting responsibilities; nervous habits (e.g. nail biting, pacing); using alcohol, cigarettes, or drugs to relax. The term preferably covers stress which has not been diagnosed as a major heath issue by a qualified health care provider or physician. However, the term may also include stress which has been diagnosed as a major heath issue by a qualified health care provider or physician, for example a Trauma- and Stressor-Related Disorder as defined in the DSM-5.

The “symptoms of insomnia” include finding it hard to go to sleep; frequent waking during the night; problems returning to sleep after awakenings; waking up early and being unable to go back to sleep; still feeling tired after waking up; finding it hard to nap during the day even though the subject is tired; feeling tired and irritable during the day. The term preferably covers insomnia which has not been diagnosed as a major heath issue by a qualified health care provider or physician. However, the term may also include insomnia which has been diagnosed as a major heath issue by a qualified health care provider or physician, for example insomnia or an insomnia disorder as defined in the International Classification of Sleep Disorders, 3rd edition (ICSD-3) and the Diagnostic and Statistical Manual for Mental Disorders, 5th edition (DSM-5).

In one embodiment, the subject does not have a current diagnosis of a mental disorder corresponding to the DSM-5 criteria for: depressive disorders, trauma- and stressor-related disorders, obsessive-compulsive and related disorders. In one embodiment, the subject does not have a current diagnosis of a mental disorder corresponding to the DSM-5 criteria for anxiety disorders. In this embodiment, the subjects may have had a previous diagnosis of the relevant DSM-5 mental disorder, but the diagnosis is not current at the time of treatment by the present method.

Preferably the anxiety is not associated with obsessive-compulsive disorder, acute stress disorder, or posttraumatic stress disorder.

Preferably the subject has symptoms of anxiety, stress and/or depression secondary to a medical condition, more preferably a chronic medical condition. The medical condition may be one of the following: cancer, cancer treatment, chronic diseases such as heart and lung disease or diabetes, migraine, insomnia or sleep disorder, chronic pain; gastrointestinal disorder; neurological disorder, osteoarthritis or inflammatory arthritis. The medical condition may be mental illness that is not a mental disorder corresponding to the DSM-5 criteria for: depressive disorders, anxiety disorder trauma- and stressor-related disorders, obsessive-compulsive and related disorders.

Cannabidiol, as used herein, refers to 2-[3-methyl-6-(1-methylethenyl)-2-cyclohexen-1-yl]-5-pentyl-1,3-benzenediol. The synthesis of cannabidiol is described, for example, in Petilka et al., Helv. Chim.Acta, 52: 1102 (1969) and in Mechoulam et al., J. Am. Chem. Soc., 87:3273 (1965), which are hereby incorporated by reference.

The term “cannabidiol” may include pre-cursors of cannabidiol such as cannabidiolic acid (CBDA or CBD-A). In one form of the invention the term “cannabidiol” only covers cannabidiol per se. In another form of the invention, the term “cannabidiol” covers both cannabidiol and precursor compounds. In another form of the invention, the term “cannabidiol” covers both cannabidiol and the precursor compound, cannabidiolic acid.

Preferably the composition comprises between 50 mg and 200 mg cannabidiol. More preferably the composition comprises between 50 mg and 150 mg cannabidiol. The composition may comprise between 60 mg and 200 mg, 60 mg and 150 mg, 70 mg and 200 mg, 70 mg and 150 mg, 80 mg and 200 mg, 90 mg and 200 mg, 90 mg and 150 mg, 100 mg and 200 mg, 100 mg and 150 mg.

The composition may comprise 50 mg, 60 mg, 70 mg, 80 mg, 90 mg, 100 mg, 110 mg, 120 mg, 130 mg, 140 mg, 140 mg, 150 mg, 160 mg, 170 mg, 180 mg, 190 mg, 200 mg. More preferably the composition comprises 60 mg cannabidiol.

The dose of cannabidiol being administered to the subject may be increased from a lower dose to a higher dose over a time period. For example, the subject may be first administered a dose of 50 mg per day. Over the course of several weeks, this dose may be increased to 60 mg, or 100 mg, or 150 mg. The increase is dosage may be carried out for a range of reasons, including a wish to avoid side-effects caused by initial administration of high doses of cannabidiol to a subject unused to such doses, and increasing the dose if the subject is not responding sufficiently to the initial lower dose.

In addition to cannabidiol, the compositions may contain more than one cannabinoid. For example, the composition of the present invention may contain a combination of two, three or more cannabinoids.

Preferably the compositions comprise a majority of one cannabinoid. More preferably, the composition may comprise at least 80%, 81%, 82%, 83%, 84%, 85%, 86%, 87%, 88%, 89%, 90%, 91%, 92%, 93%, 94%, 95%, 96%, 97%, 98%, 99% of one cannabinoid. The remaining % of the composition may be other cannabinoids or other impurities. Preferably the majority cannabinoid is cannabidiol (CBD).

For example, the composition may comprise greater than or equal to 98% (w/w) CBD and less than or equal to 2% (w/w) other cannabinoids. The less than or equal to 2% (w/w) other cannabinoids may comprise the cannabinoids tetrahydrocannabinol (THC); cannabidivarin (CBDV); cannabinol (CBN); cannabigerol (CBG); tetrahydrocannabivarin; (THCV); cannabichromene (CBC); nantradol hydrochloride; nabilone and other naturally occurring cannabinoids.

Suitable pharmaceutically acceptable salts of cannabinoids for which a salt can be made include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino) ethane salt, monomethyl-monoethanolamine salt, procaine salt and caffeine salt), a basic amino acid salt (such as arginine salt and lysine salt), tetraalkyl ammonium salt and the like, or other salt forms that enable the cannabinoid to remain soluble in a liquid medium, or to be prepared and/or effectively administered in a liquid medium, preferable an aqueous medium. The above salts may be prepared by a conventional process, for example from the corresponding acid and base or by salt interchange.

Examples of suitable pharmaceutically acceptable salts of cannabinoids for which a salt can be made include inorganic acid addition salts such as hydrochloride, hydrobromide, sulfate, phosphate, and nitrate; organic acid addition salts such as acetate, propionate, succinate, lactate, glycolate, malate, tartrate, citrate, maleate, fumarate, methansulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

In one embodiment at least a portion of at least one of the cannabinoids present in the composition is isolated from cannabis plant material. Preferably at least a portion of the CBD present in the composition is isolated from cannabis plant material.

In a further embodiment of the invention substantially all of at least one of the cannabinoids present in the composition is isolated from cannabis plant material. Preferably substantially all of the CBD present in the composition is isolated from cannabis plant material.

In a further embodiment of the invention substantially all of the cannabinoids present in the composition are isolated from cannabis plant material.

Preferably the cannabis plant material is from Cannabis sativa, Cannabis indica, or Cannabis ruderalis plants. Preferably the cannabis plant is a high-CBD containing cannabis chemotype.

In a further embodiment of the invention at least a portion of at least one of the cannabinoids present in the composition is prepared synthetically. Preferably at least a portion of the CBD present in the composition is prepared synthetically.

In a further embodiment of the invention substantially all of at least one of the cannabinoids present in the composition is prepared synthetically. Preferably substantially all of the CBD present in the composition is prepared synthetically.

Preferably substantially all of the cannabinoids present in the composition are prepared synthetically.

If at least a portion or substantially all of the CBD present in the composition is synthetic, then preferably the synthetic CBD only contains the (—) CBD enantiomer.

The composition may contain both cannabinoids isolated from cannabis plant material and synthetically prepared cannabinoids. For example, the composition may contain synthetically prepared CBD and a mixture of other cannabinoids isolated from cannabis plant material.

Preferably the composition comprises not more than 2.0% or 1.5% (w/w) THC based on total amount of cannabinoid in the preparation. More preferably the composition comprises about 0.01% to about 0.1% (w/w) THC based on total amount of cannabinoid in the preparation. More preferably the composition comprises about 0.02% to about 0.05% (w/w) THC based on total amount of cannabinoid in the preparation. More preferably the composition comprises 0% (w/w) THC.

### Treatment and Prevention

In certain embodiments the administration of cannabidiol is expected to treat or prevent one or more symptoms of psychological distress in a subject wherein the subject has symptoms of anxiety, stress or depression or insomnia. For example, the cannabidiol may reduce the incidence and/or severity of episodes of anxiety, stress or depression or insomnia. Therapeutic effects of the present invention include, but are not limited to, reduction in frequency and/or severity of episodes of anxiety, stress or depression or insomnia.

The present invention may treat the symptoms of anxiety, stress or depression or insomnia by reducing the incidence and/or severity of one or more of the symptoms of anxiety, stress or depression or insomnia, such as those listed above in relation to “symptoms of anxiety”, “symptoms of depression”, “symptoms of stress” and/or “symptoms of insomnia”. The present invention may prevent the onset of one or more of the symptoms of anxiety, stress or depression or insomnia, such as those listed above in relation to “symptoms of anxiety”, “symptoms of depression”, “symptoms of stress” and/or “symptoms of insomnia”.

In certain embodiments, the administration of cannabidiol is expected to improve the symptoms of anxiety, stress or depression or insomnia. By “improve”, it is meant that there is a reduction in frequency and/or severity of episodes of anxiety, stress or depression or insomnia.

Preferably, the treatment reduces one or more symptoms of psychological distress in a subject wherein the subject has symptoms when assessed using a validated scale by a drop from a higher score or category to a score or lower category for at least one of anxiety, stress or depression or insomnia. In this respect, it does not matter what the original score or category was in the validated scale, merely that the treatment has reduced that score or category to a lower score or category. More preferably, the treatment reduces the symptoms of anxiety, stress or depression by a drop from a higher score or category to a lower score or category for at least two of anxiety, stress or depression. For example, if DASS is being used, the treatment may reduce the anxiety score from a category of “very severe” to “severe”, or reduce the depression score from “moderate” to “mild”. The treatment further preferably reduces the symptoms of insomnia by a drop from a higher score or category to a lower score or category. For example, if 151 is being used, the treatment may reduce the insomnia score from a category of “severe” to “moderate”.

Preferably, the subject being treated scores at least:
a) 8 for anxiety (mild-moderate);
b) 15 for stress (mild-moderate); and/or
c) 10 for depression (mild-moderate)
   on at least one of the categories of anxiety, stress or depression when assessed by the Depression Anxiety Stress Scale (DASS-21 or DASS-42); and the treatment reduces the subject's DASS score on at least one of the categories of anxiety, stress or depression.

Preferably, the subject being treated scores at least 8 for insomnia when assessed by the Insomnia Severity Index (ISI) 7-item self-report questionnaire; and the treatment reduces the subject's ISI score.

As the symptoms of psychological distress include anxiety, stress or depression, and can also include insomnia, it is preferred that a reduction in scores for one set of symptoms is associated with a reduction in scores for a second set of symptoms. For example, without being held to any theory, as the symptoms of anxiety, stress and/or depression may lead to symptoms of insomnia and equally the symptoms of insomnia may lead to symptoms of anxiety, stress and/or depression, it is preferred that reduction in one or more of the symptoms results in a reduction in at least one other symptom. For example, reducing the symptoms of anxiety may assist in reducing the symptoms of insomnia, or reducing the symptoms of insomnia may assist in reducing the symptoms of stress.

The reduction in the symptoms of psychological distress of anxiety, stress or depression or insomnia may not occur immediately after administration of the first dose of cannabidiol. The reduction in the symptoms of psychological distress may take days, weeks or months to be detectable by use of a validated scale. In one embodiment, the reduction in the symptoms of anxiety, stress or depression is detectable by a drop from a higher score or category to a score or lower category for at least one of anxiety, stress or depression over a time period of one week, two weeks, three weeks or one month.

Preferably, the reduction in the symptoms of anxiety, stress or depression is detectable by a drop from a higher score or category to a score or lower category for at least one of anxiety, stress or depression or insomnia is maintained for at least as long as the cannabidiol is being administered. For example, the reduction in the symptoms of anxiety, stress or depression or insomnia may be maintained for at least 1 month, 2 months, 6 months, 12 months, 2 years or indefinitely whilst the cannabidiol is being administered. In addition, the reduction in the symptoms of anxiety, stress or depression or insomnia may be maintained for at least 1 month, 2 months, 6 months, 12 months, 2 years or indefinitely after administration of the last dose of cannabidiol.

The composition may be administered daily for at least one week, two weeks, three weeks, one month. The daily administration may be continued for two months, three months, four months, five months, six months, seven months, eight months, nine months, ten months, eleven months or twelve months. The daily delivery may be administered for one week, two weeks, three weeks, one month or a few months, then there may be a break in delivery for several months, until symptoms of psychological distress recur and treatment is resumed.

A primary advantage of the present invention is expected to be the improvement in the mental wellbeing of the subject with a reduction or absence in the typical side effects of conventional therapies such as antidepressant medications (including SSRIs and SNRIs), beta blockers and benzodiazepines.

Those skilled in the art will appreciate that the term “dosage form” or “unit dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The exact regimen for administration of the cannabidiol may depend on the needs of the individual subject being treated, the type of treatment administered, and/or the judgment of the attending medical specialist. As used herein, the terms “subject” and “patient” includes both humans and animals. In some embodiments, the subject or patient is a human adult, human adolescent, human child, or human infant. As those skilled in the art will appreciate, the dosage administered will depend upon the condition being treated, the age, health and weight of the recipient, the type of concurrent treatment, if any, and the frequency of treatment. In some embodiments, a composition or a pharmaceutical composition comprising CBD may be administered in a therapeutically effective amount. A therapeutically effective amount may be administered according to a dosing regimen comprising one or more unit doses. Generally, a therapeutically effective amount is sufficient to achieve a benefit to the subject (e.g., prophylaxis, treating, modulating, curing, preventing and/or ameliorating a disease or disorder).

A therapeutically effective amount (and/or unit dose) of a composition for any particular subject may depend upon a variety of factors including the disease or disorder being treated; disease or disorder severity; the activity of the specific composition employed; the specific composition employed; the age; body weight; fitness; comorbid conditions (e.g., other than the diseases or disorder(s) being treated) general health; sex; and diet of the patient; personal history; genetic characteristic; lifestyle parameter; severity of cardiac defect and/or level of risk of cardiac defect; the time of administration; route of administration; concomitant treatments or medications; and/or rate of excretion or metabolism of the specific composition employed; the duration of the treatment; combinations thereof; as well as other factors well known in the medical arts. In view of the present disclosure, one of ordinary skill in the art will be readily able to determine appropriate dosages depending on these and other related factors. In addition, both objective and subjective assays may optionally be employed to identify optimal dosage ranges. In some particular embodiments, appropriate doses or amounts to be administered may be extrapolated in view of the instant disclosure from dose-response curves derived from in vitro or animal model test systems.

The present invention contemplates dosing regiments comprising single as well as multiple administrations of a composition as described. The composition can be administered at regular intervals, depending on the nature, severity and extent of the subject's condition. In some embodiments, the composition may be administered periodically at regular intervals (e.g., once every year, once every six months, once every five months, once every three months, bimonthly (once every two months), monthly (once every month), biweekly (once every two weeks), weekly, daily, multiple times each day, or continuously).

A therapeutically effective amount of cannabidiol may be administered according to a dosing regimen that may comprise multiple unit doses. For any particular composition, a therapeutically effective amount (and/or an appropriate unit dose within an effective dosing regimen) may vary, for example, depending on route of administration, or combination with other pharmaceutical agents.

In some embodiments, the composition as described may be administered as a single dose. In some embodiments, a composition or a pharmaceutical composition as described may be administered at regular intervals. Administration at an “interval,” as used herein, indicates that the therapeutically effective amount is administered periodically (as distinguished from a one-time dose). The interval can be determined by standard clinical techniques. In some embodiments, the composition as described may be administered bimonthly, monthly, twice monthly, triweekly, biweekly, weekly, twice weekly, thrice weekly, daily, twice daily, every six hours, every four hours, every two hours, or hourly. The administration interval for a given individual need not be a fixed interval, but may be varied over time, depending on the needs of the individual.

In some embodiments, the composition as described is administered at regular intervals indefinitely. In some embodiments, the composition is administered at regular intervals for a defined period.

It is to be further understood that for any particular subject, specific dosage regimens should be adjusted over time according to the individual need and the professional judgment of the person administering or supervising the administration of the cannabidiol and that dosage ranges set forth herein are exemplary only and are not intended to limit the scope or practice of the claimed invention.

As used herein, “improve”, “increase”, “inhibit” or “reduce”, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

“Prevent or prevention” as used herein when used in connection with the occurrence of anxiety, stress or depression, refers to reducing the risk of developing anxiety, stress or depression and/or to delaying onset of one or more characteristics or symptoms of anxiety, stress or depression. Prevention may be considered complete when onset of anxiety, stress or depression has been delayed for a predefined period of time.

According to the present invention, “symptoms are reduced” when one or more symptoms of anxiety, stress or depression is reduced in magnitude (e.g., intensity, severity, etc.) and/or frequency. For purposes of clarity, a delay in the onset of a particular symptom is considered one form of reducing the frequency of that symptom.

As used herein, the term “treat”, “treatment”, or “treating” refers to any method used to partially or completely alleviate, ameliorate, relieve, inhibit, prevent, delay onset of, reduce severity of, and/or reduce incidence of one or more symptoms or features of anxiety, stress or depression. In some embodiments, treatment refers to administration of a therapy that partially or completely alleviates, ameliorates, relives, inhibits, delays onset of, reduces severity of, and/or reduces incidence of one or more symptoms, features, and/or causes of anxiety, stress or depression. Treatment may be administered to a subject who does not exhibit signs of anxiety, stress or depression. In some embodiments, treatment may be administered to a subject who exhibits only early signs of anxiety, stress or depression, for example for the purpose of decreasing the risk of developing pathology associated with anxiety, stress or depression.

As used herein, the term “subject” or “patient” refers an organism, typically a mammal (e.g., a human). In some embodiments, a subject or patient refers to any organism (e.g., mammals such as mice, rats, rabbits, non-human primates, and humans; insects; worms; etc.) to which a provided compound or composition is administered in accordance with the present disclosure e.g., for experimental, diagnostic, prophylactic, and/or therapeutic purposes. In some embodiments, a subject is suffering from anxiety, stress or depression or insomnia. In some embodiments, a subject has symptoms associated with anxiety, stress or depression or insomnia. In some embodiments, a subject is susceptible to anxiety, stress or depression or insomnia or symptoms associated with anxiety, stress or depression or insomnia. In some embodiments, a subject displays one or more symptoms or characteristics of anxiety, stress or depression or insomnia. In some embodiments, a subject does not display any symptoms or characteristics of anxiety, stress or depression or insomnia. In some embodiments, a subject is someone with one or more features characteristic of susceptibility to or risk of anxiety, stress or depression or insomnia. In some embodiments, a subject is a patient. In some embodiments, a subject is an individual to whom diagnosis and/or therapy is and/or has been administered. “Individual”, “patient” and “subject” are used interchangeably.

### Composition

According to one aspect of the invention, there is provided a composition comprising between 50 mg and 200 mg cannabidiol for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and wherein the total daily dose of cannabidiol is between 50 mg/day and 200 mg/day.

There is further provided a composition comprising between 50 mg and 200 mg cannabidiol for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia and wherein the total daily dose of cannabidiol is between mg/day and 200 mg/day.

Preferably, the subject has symptoms of two or more of anxiety, stress and/or depression. Preferably the method reduces the subject's score on the validated scale for anxiety or reduces the anxiety biomarker. Preferably the method also reduces the subject's score on the validated scale for stress and/or depression or reduces the stress and/or depression biomarker(s). Preferably the method reduces the subject's score on the validated scale for anxiety and/or reduces the subject's score on the validated scale for stress and/or reduces the subject's score on the validated scale for depression. Preferably the method reduces the subject's score on the validated scale for insomnia.

The composition may be delivered once per day, or more than once per day (for example, twice per day, three times per day or four times per day) for a total daily dose of between 50 mg and 200 mg of cannabidiol.

The composition may be administered orally, by nasal or pulmonary administration, or administered via the oral mucosa.

### Delivery

In one embodiment of the invention, cannabidiol is administered to the subject using a dosing regimen selected from the group consisting of: three times daily; two times daily; daily; every second day, every third day, once weekly; once fortnightly and once monthly.

In accordance with certain embodiments, the composition is administered regularly until treatment is obtained. In one preferred embodiment, the composition is administered to the subject in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention. Preferably, the composition of the treatment regimen is administered to the subject between one and five times per day, more preferably once or twice per day.

The composition may be administered daily for at least one week, two weeks, three weeks or one month. The daily administration may be continued for two months, three months, four months, five months, six months, seven months, eight months, nine months, ten months, eleven months or twelve months. The daily delivery may be administered for one or a few months, then there may be a break in delivery for several months, until symptoms of psychological distress recur and treatment is resumed.

The compositions of the present invention may be delivered by via oral delivery, delivery via the oral mucosa (sublingual or buccal absorption), or via nasal or pulmonary delivery.

### Treatment by Oral Administration

In one aspect, the composition used in the method for the treatment or prevention of psychological distress in a subject is an oral pharmaceutical composition comprising cannabidiol. The oral treatment method may comprise the administration of cannabidiol to the gastrointestinal (GI) tract of the subject. Preferably, the cannabidiol enters the blood stream via absorption in the GI tract and is systemically available to the subject.

### Treatment by Oral Mucosal Administration

In one aspect, the composition used in the method for the treatment or prevention of psychological distress in a subject in need of such treatment is an oral mucosal pharmaceutical composition comprising cannabidiol. The nasal or pulmonary treatment method may comprise the administration of cannabidiol to the oral mucosa (sublingual or buccal mucosa) of the subject. The cannabidiol may enter the blood stream via absorption from the sublingual or buccal mucosa and be systemically available to the subject.

### Treatment by Nasal or Pulmonary Administration

In one aspect, the composition used in the method for the treatment or prevention of psychological distress in a subject in need of such treatment is a nasal or pulmonary pharmaceutical composition comprising cannabidiol. The nasal or pulmonary treatment method may comprise the administration of cannabidiol to the nasal or pulmonary system of the subject. The cannabidiol may enter the blood stream via absorption in the nasal or pulmonary system and be systemically available to the subject.

### Excipients

The compositions of the invention may optionally include pharmaceutically acceptable nontoxic excipients and carriers. As used herein, a “pharmaceutical carrier” is a pharmaceutically acceptable solvent, suspending agent, excipient or vehicle for delivering the cannabidiol to the subject. The carrier may be liquid or solid and is selected with the planned manner of administration in mind.

The composition of the invention may be selected from the group consisting of: an immediate release composition, a delayed release composition, a controlled release composition and a rapid release composition.

The compositions described herein may be formulated by including such dosage forms in an oil-in-water emulsion, or a water-in-oil emulsion. In such a composition, the immediate release dosage form is in the continuous phase, and the delayed release dosage form is in a discontinuous phase. The composition may also be produced in a manner for delivery of three dosage forms as hereinabove described. For example, there may be provided an oil-in-water-in-oil emulsion, with oil being a continuous phase that contains the immediate release component, water dispersed in the oil containing a first delayed release dosage form, and oil dispersed in the water containing a third delayed release dosage form.

The compositions described herein may be in the form of a liquid composition. The liquid composition may comprise a solution that includes a therapeutic agent (e.g. cannabidiol) dissolved in a solvent. Generally, any solvent that has the desired effect may be used in which the therapeutic agent dissolves and which can be administered to a subject. Generally, any concentration of therapeutic agent that has the desired effect can be used. The composition in some variations is a solution which is unsaturated, a saturated or a supersaturated solution. The solvent may be a pure solvent or may be a mixture of liquid solvent components. In some variations the solution formed is an in-situ gelling composition. Solvents and types of solutions that may be used are well known to those versed in such drug delivery technologies.

The composition may or may not contain water. Preferably, the composition does not contain water, i.e. it is non-aqueous. In another preferred embodiment, the composition does not comprise a preservative.

The pharmaceutical composition may be formulated according to the conventional pharmaceutical practice (see, for example, Remington: The Science and Practice of Pharmacy, 20th edition, 2000, ed; A. R. Gennaro, Lippincott Williams & Wilkins, Philadelphia, and Encyclopedia of Pharmaceutical Technology, eds; J. Swarbrick and J. C. Boylan, 1988-1999, Marcel Dekker, New York; Remington's Pharmaceutical Sciences, 18^{th} Edition, Mack Publishing Company, Easton, Pennsylvania, USA).

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

The composition may be in the form of a controlled-release composition and may include a degradable or non-degradable polymer, hydrogel, organogel, or other physical construct that modifies the release of the cannabidiol. It is understood that such compositions may include additional inactive ingredients that are added to provide desirable colour, stability, buffering capacity, dispersion, or other known desirable features. Such compositions may further include liposomes, such as emulsions, foams, micelles, insoluble monolayers, liquid crystals, phospholipid dispersions, lamellar layers and the like. Liposomes for use in the invention may be formed from standard vesicle-forming lipids, generally including neutral and negatively charged phospholipids and a sterol, such as cholesterol.

### Oral Compositions

Contemplated for use herein are oral solid dosage forms, which are described generally in Martin, Remington's Pharmaceutical Sciences, 18th Ed. (1990 Mack Publishing Co. Easton PA 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the composition will include therapeutic agents (e.g. cannabidiol), and inert ingredients which allow for protection against the stomach environment, and release of the cannabidiol in the intestine.

For the cannabidiol of the invention, the location of release may be the stomach, the small intestine (the duodenum, the jejunum, or the ileum), or the large intestine. One skilled in the art has available compositions that will not dissolve in the stomach, yet will release the material in the duodenum or elsewhere in the intestine. In one aspect, the release will avoid the deleterious effects of the stomach environment, either by protection of the composition or by release of the cannabidiol beyond the stomach environment, such as in the intestine.

It is believed that the oral bioavailability of cannabinoids is only 4% to 12% and absorption is highly variable. Although most cannabinoids are generally easily absorbed due to their high partition coefficient (P), they are subject to degradation in the stomach and significant first-pass metabolism. Therefore, preferably, the cannabidiol of the present invention is released in the lower gastrointestinal tract.

The oral dosage method may be provided using an oral sustained release pharmaceutical composition comprising a therapeutically effective pharmaceutical composition according to the invention, and a release retardant.

In one aspect of the present invention the release retardant is a water-soluble, water swellable and/or water insoluble polymer. In particular, water-soluble polymers may be selected from the group comprising ethylcellulose, hydroxypropyl cellulose, hydroxypropyl methyl cellulose. The release retardant may be an enteric coating or a semipermeable membrane. In another aspect of the invention the release retardant is a non-polymeric release retardant. More particularly, the non-polymeric release retardant may be hydrogenated castor oil. The compositions of the invention may be milled or granulated and compressed into tablets or encapsulated into capsules according to conventional procedures known in the art.

To ensure full gastric resistance, a coating impermeable to at least pH 5.0 is used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This includes without limitation sugar coatings, or coatings that make the tablet easier to swallow. Exemplary capsules include hard shell capsules (such as gelatine) for delivery of dry therapeutics such as powders, or soft gelatine shells for liquid therapeutic forms. The shell material of cachets in certain aspects is thick starch or other edible paper. For pills, lozenges, moulded tablets or tablet triturates, moist massing techniques are also contemplated, without limitation.

As used herein, the term “sustained release” means the gradual but continuous or sustained release over a relatively extended period of the therapeutic compound content after oral ingestion. The release may continue after the pharmaceutical composition has passed from the stomach and through until and after the pharmaceutical composition reaches the intestine. The phrase “sustained release” also means delayed release wherein release of the therapeutic compound is not immediately initiated upon the pharmaceutical composition reaching the stomach but rather is delayed for a period of time, for example, until when the pharmaceutical composition reaches the intestine. Upon reaching the intestine, the increase in pH may then trigger release of the therapeutic agent from the pharmaceutical composition.

Though term “release retardant” is used herein, means a substance that reduces the rate of release of a therapeutic agent from a pharmaceutical composition when orally ingested. The release retardant may be a polymer or a non-polymer. The release retardant may be used according to any one of several sustained release systems including, for example, a diffusion system, a dissolution system and/or an osmotic system.

In certain aspects, the therapeutic agent (e.g. cannabidiol) is included in the composition as fine multi-particulates in the form of granules or pellets of particle size about 1 mm. The composition of the material for capsule administration is, in certain aspects, a powder, lightly compressed plug, or even as a tablet. In one aspect, the composition comprising the therapeutic agent could be prepared by compression.

Colourants and flavouring agents may optionally be included. For example, compositions may be formulated (such as, and without limitation, by liposome or microsphere encapsulation) and then further contained within an edible product, such as a refrigerated beverage containing colorants and flavouring agents.

The volume of the composition may be diluted or increased with an inert material. These diluents could include carbohydrates, especially mannitol, alpha-lactose, anhydrous lactose, cellulose, sucrose, modified dextrans and starch. Certain inorganic salts are also optionally used as fillers including calcium triphosphate, magnesium carbonate and sodium chloride. Some commercially available diluents are Fast-Flo, Emdex, STA-Rx 1500, Emcompress and Avicell.

In other embodiments, disintegrants are included in the solid dosage form compositions of the present invention. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, Karaya or tragacanth. Alginic acid and its sodium salt are also useful as disintegrants.

Binders are contemplated to hold the cannabidiol together to form a hard tablet and include, without limitation, materials from natural products such as acacia, tragacanth, starch and gelatine. Other binders include, without limitation, methylcellulose (MC), ethyl cellulose (EC) and carboxymethyl cellulose (CMC). Polyvinyl pyrrolidone (PVP) and hydroxypropylmethyl cellulose (HPMC) are contemplated for use in alcoholic solutions to granulate the therapeutic.

An antifrictional agent may be optionally included in the compositions of the invention to prevent sticking during the composition process. Lubricants may be optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants may also be used such as include sodium lauryl sulfate, magnesium lauryl sulfate, polyethylene glycol of various molecular weights, and Carbowax 4000 and 6000.

Glidants that might improve the flow properties of the compound during composition and to aid rearrangement during compression might be optionally added. The glidants may include without limitation starch, talc, pyrogenic silica and hydrated silicoaluminate.

To aid dissolution of the therapeutic agent (e.g. cannabidiol) into the aqueous environment, a surfactant might be added in certain embodiments as a wetting agent. Surfactants may include, for example and without limitation, anionic detergents such as sodium lauryl sulfate, dioctyl sodium sulfosuccinate and dioctyl sodium sulfonate. Cationic detergents might be optionally used and could include, without limitation, benzalkonium chloride or benzethomium chloride. The list of potential nonionic detergents that could be included in the composition as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the composition of the therapeutic agent either alone or as a mixture in different ratios.

Additives which that potentially enhance uptake of the therapeutic agent include, without limitation, the fatty acids oleic acid, linoleic acid and linolenic acid.

Controlled release composition may be desirable. In certain aspects, the therapeutic agents could be incorporated into an inert matrix that permits release by either diffusion or leaching mechanisms i.e., gums. In some aspects, slowly degenerating matrices may also be incorporated into the composition. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some enteric coatings also have a delayed release effect.

In other aspects, a mix of materials might be used to provide the optimum film coating. Film coating may be carried out, for example, in a pan coater or in a fluidized bed or by compression coating.

### Nasal and Pulmonary Compositions

Compositions of the invention may be administered via nasal or pulmonary delivery. Preferably, the nasal or pulmonary compositions comprise cannabidiol.

A wide range of mechanical devices designed for pulmonary delivery of therapeutic agents exist, including but not limited to nebulizers, metered-dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Missouri; the Acorn II nebulizer, manufactured by Marquest Medical Products, Englewood, Colorado; the Ventolin metered dose inhaler, manufactured by Glaxo Inc., Research Triangle Park, North Carolina; and the Spinhaler powder inhaler, manufactured by Fisons Corp., Bedford, Massachusetts.

All such devices require the use of compositions suitable for the dispensing of the cannabidiol. Typically, each composition is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Compositions suitable for use with a nebulizer, either jet or ultrasonic, will typically comprise the cannabidiol suspended in water or a non-aqueous solvent. The composition may also include a buffer and a simple sugar (e.g., for stabilization and regulation of osmotic pressure). The nebulizer composition may also contain a surfactant, to reduce or prevent surface induced aggregation of the cannabidiol caused by atomization of the solution in forming the aerosol.

Compositions for use with a metered dose inhaler device will generally comprise a finely divided powder containing the cannabidiol suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Compositions for dispensing from a powder inhaler device will comprise a finely divided dry powder containing the cannabidiol and may also include a bulking agent, such as lactose, sorbitol, sucrose, or mannitol in amounts which facilitate dispersal of the powder from the device, e.g., 50 to 90% by weight of the composition. The cannabidiol should most advantageously be prepared in particulate form with an average particle size of less than 10 microns, most preferably 0.5 to 5 microns, for most effective delivery to the distal lung.

Nasal delivery of cannabidiol in the treatment methods of the present invention is also contemplated. Nasal delivery allows the passage of the cannabidiol to the blood stream directly after administering the therapeutic product to the nose, without the necessity for deposition of the cannabidiol in the lung. Compositions for nasal delivery include those with dextran or cyclodextran.

### Oral Mucosal Compositions

The cannabidiol of the present invention may be delivered via the oral mucosal surfaces, such as the buccal mucosa or the sublingual mucosa.

The oral mucosal composition may comprise one or more pharmaceutical excipients. The excipient can be one or more selected from the group consisting of diluents, sweeteners, viscosity enhancing agents, dispersing agents, preservatives, flavouring agents and the like. One excipient can perform more than one function. In one embodiment, the one or more pharmaceutical excipients include a lubricant and/or a diluent.

Dispersing agents include, but are not limited to, colloidal silicon dioxide and surfactants, wherein the surfactant is used alone or as an admixture with one or more surfactant. In one embodiment, the dispersing agent can be any dispersing agent known in the art. Combinations of colloidal silicon dioxide with one or more surfactants can also be used.

In one embodiment, the lubricant can be any lubricant known in the art. Non-limiting examples of lubricants include magnesium stearate, calcium stearate, stearic acid, glyceryl behenate, hydrogenated vegetable oil, and glycerine fumarate, and/or a combination thereof. In another embodiment, the lubricant is selected from magnesium stearate, calcium stearate, stearic acid, glyceryl behenate, hydrogenated vegetable oil, and glycerine fumarate. In another embodiment, the lubricant is calcium stearate. In yet another embodiment, the lubricant is stearic acid. In further embodiment, the lubricant is magnesium stearate.

In one embodiment, the diluent can be any diluent known in the art. Non-limiting examples of diluents include starch, pregelatinized starch, microcrystalline cellulose, calcium carbonate, dibasic calcium phosphate, tribasic calcium phosphate, calcium phosphate, lactose, dextrose, fructose, lactitol, lactose, magnesium carbonate, magnesium oxide, maltitol, maltodextrin, maltose, simethicone, sodium chloride, talc, xylitol, sorbitol, mannitol, and sucrose, and/or a combination thereof. In another embodiment, the diluent is selected from starch, pregelatinized starch, microcrystalline cellulose, calcium phosphate, lactose, sorbitol, mannitol, and sucrose. In another embodiment, the diluent is calcium phosphate. In yet another embodiment, the diluent is mannitol. In further embodiment, the diluent is microcrystalline cellulose.

### Use

According to another aspect of the invention, there is provided the use of a composition comprising between 50 mg and 200 mg cannabidiol for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and wherein the total daily dose of cannabidiol is between 50 mg/day and 200 mg/day and wherein the composition is administered for at least one week.

According to another aspect of the invention, there is provided the use of between mg and 200 mg cannabidiol, in the manufacture of composition for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and wherein the total daily dose of cannabidiol is between 50 mg/day and 200 mg/day and wherein the composition is administered for at least one week.

According to another aspect of the invention, there is provided the use of a composition comprising between 50 mg and 200 mg cannabidiol for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia and wherein the total daily dose of cannabidiol is between 50 mg/day and 200 mg/day and wherein the composition is administered for at least one week.

According to another aspect of the invention, there is provided the use of between mg and 200 mg cannabidiol, in the manufacture of composition for the treatment or prevention of one or more symptoms of psychological distress in a subject wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia and wherein the total daily dose of cannabidiol is between 50 mg/day and 200 mg/day and wherein the composition is administered for at least one week.

Preferably, the subject has symptoms of two or more of anxiety, stress and/or depression. Preferably the method reduces the subject's score on the validated scale for anxiety or reduces the anxiety biomarker. Preferably the method also reduces the subject's score on the validated scale for stress and/or depression or reduces the stress and/or depression biomarker(s). Preferably the method reduces the subject's score on the validated scale for anxiety and/or reduces the subject's score on the validated scale for stress and/or reduces the subject's score on the validated scale for depression.

Preferably the method reduces the subject's score on the validated scale for insomnia or reduces the insomnia biomarker.

The composition may be delivered once per day, or more than once per day (for example, twice per day, three times per day or four times per day) for a total daily dose of between and 200 mg of cannabidiol.

### Kits

According to one aspect of the invention, there is provided a kit for the treatment or prevention of one or more symptoms of psychological distress comprising:
a) a composition comprising between 50 mg and 200 mg of cannabidiol;
b) instructions for use
   wherein the subject has symptoms of at least one of anxiety, stress and/or depression when assessed by a validated scale or biomarker for identifying symptoms of anxiety, stress and/or depression and wherein the total daily dose of cannabidiol is between 50 mg/day and 200 mg/day and wherein the composition is administered for at least one week.

There is further provided a kit for the treatment or prevention of one or more symptoms of psychological distress comprising:
a) a composition comprising between 50 mg and 200 mg of cannabidiol;
b) instructions for use
   wherein the subject has symptoms of at least one of anxiety, stress and/or depression when assessed by a validated scale or biomarker for identifying symptoms of anxiety, stress and/or depression and the subject has insomnia as assessed by a validated scale or biomarker for identifying symptoms of insomnia and wherein the total daily dose of cannabidiol is between 50 mg/day and 200 mg/day and wherein the composition is administered for at least one week.

Preferably, the subject has symptoms of two or more of anxiety, stress and/or depression. Preferably the method reduces the subject's score on the validated scale for anxiety or reduces the anxiety biomarker. Preferably the method also reduces the subject's score on the validated scale for stress and/or depression or reduces the stress and/or depression biomarker(s). Preferably the method reduces the subject's score on the validated scale for anxiety and/or reduces the subject's score on the validated scale for stress and/or reduces the subject's score on the validated scale for depression.

Preferably the method reduces the subject's score on the validated scale for insomnia or reduces the insomnia biomarker.

Kits of the invention include one or more containers comprising cannabidiol as described herein, and instructions for use in accordance with any one of the methods described herein. The kit may further comprise a description for selecting a subject suitable for treatment based on identifying whether that individual has symptoms of anxiety, stress and/or depression or insomnia when assessed by a validated scale or biomarker for identifying symptoms of anxiety, stress and/or depression or insomnia. The kit may further comprise a description of administering cannabidiol as described herein to an individual at risk of developing symptoms of anxiety, stress and/or depression or insomnia when assessed by a validated scale or biomarker for identifying symptoms of anxiety, stress and/or depression or insomnia.

The composition may be delivered once per day, or more than once per day (for example, twice per day, three times per day or four times per day) for a total daily dose of between 50 mg and 200 mg of cannabidiol.

The instructions generally include information as to dosage, dosing schedule, and route of administration for the intended treatment. The containers may be unit doses, bulk packages (e.g. multi-dose packages) or sub-unit doses. Instructions supplied in the kits of the invention are typically written instructions on a label or package insert. The label or package insert indicates that the composition is used for treating and/or preventing symptoms of anxiety, stress and/or depression. Instructions may be provided for practising any of the methods described herein.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Treatment of Psychological Distress

### Methods

Detailed demographic information and validated standardised questionnaires were collected as part of routine care. Written informed consent to treatment was obtained from all patients, as well as additional consent to participate in Emyria's clinical data registry, data from participating patients were anonymised, aggregated, and analysed to establish clinical efficacy.

Clinically validated tools were used to monitor patients for management of symptoms, quality of life, physical/emotional functioning, and concomitant medication usage. Patients were asked about their cannabis history and initially pre-screened for any illicit cannabis use before commencing on medicinal cannabis treatment using the Cannabis Use Disorder Identification Test (CUDIT), a urine screen was performed to confirm negative result for cannabis in their urine before they commence treatment. After they commenced on medicinal cannabis treatment they were initially treated with low doses and monitored for safety and efficacy of treatment before dose escalation is considered.

Patients are excluded from treatment with medicinal cannabis if they: were pregnant or breast-feeding; have untreated heart disease, heart failure or arrhythmia; a history of severe psychiatric disorders; active suicidal ideation; a history of dependence on cannabis; a history of dependence or abuse of psychoactive substances; detectable urinary THC before commencement of medicinal cannabis.

### Exclusion Criteria

A potentially eligible individual who met any of the following criteria will be excluded from participation if the following criteria is met:
1. Clinically recorded cannabis drug addiction, or cannabis use disorder including individuals with scores 8 or more on the Cannabis Use Disorder Identification Test (CUDIT).
2. Untreated cardiovascular disease, arrhythmias (other than well controlled atrial fibrillation), or severe heart failure.
3. History of suicide attempts or suicidal ideation.
4. History of psychotic episode, schizophrenia or other psychotic disorders.

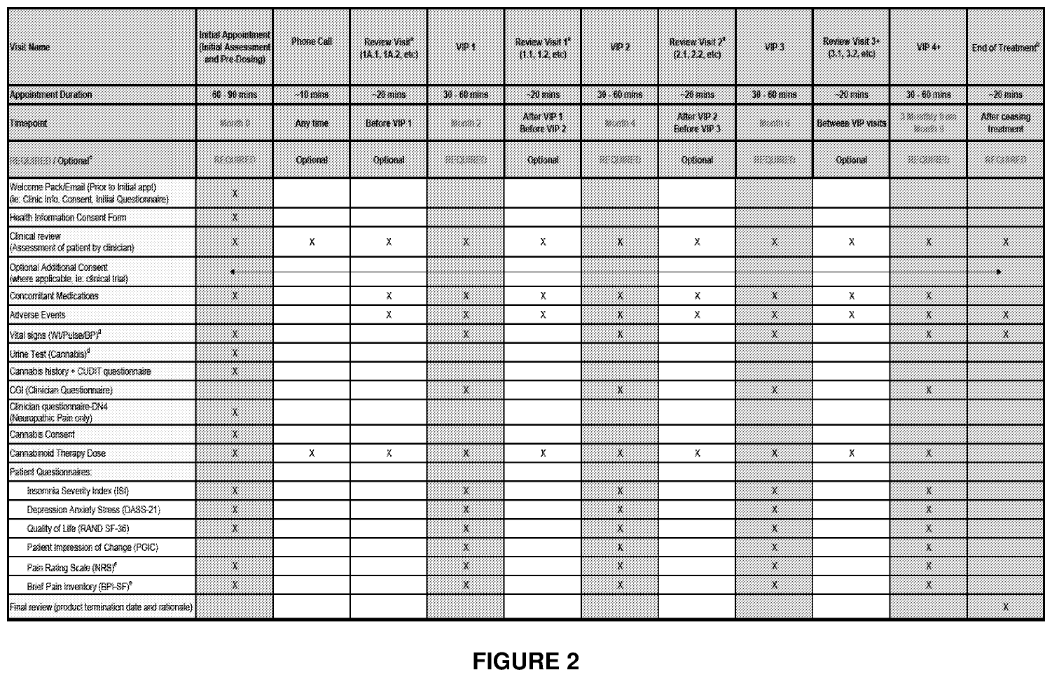

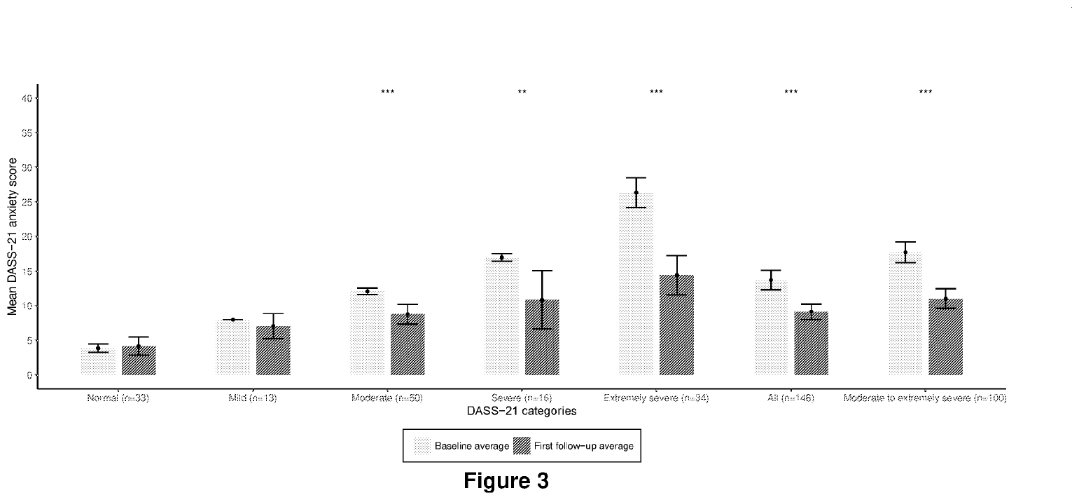

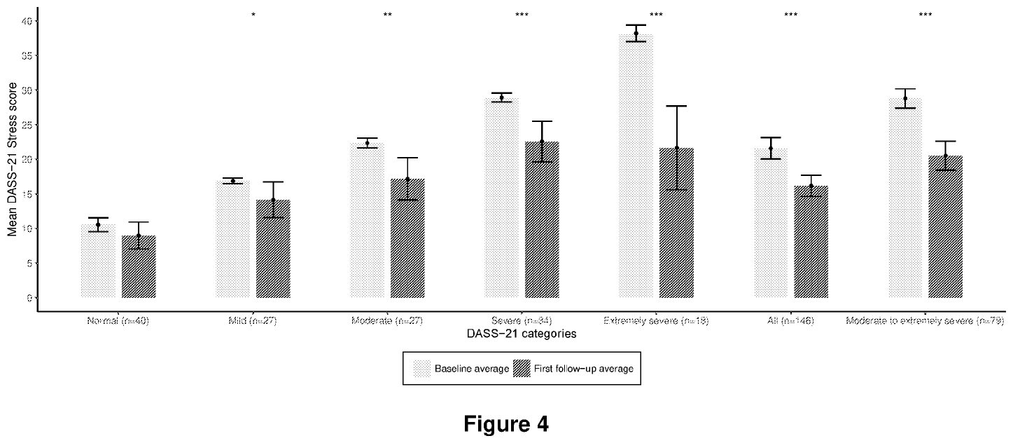

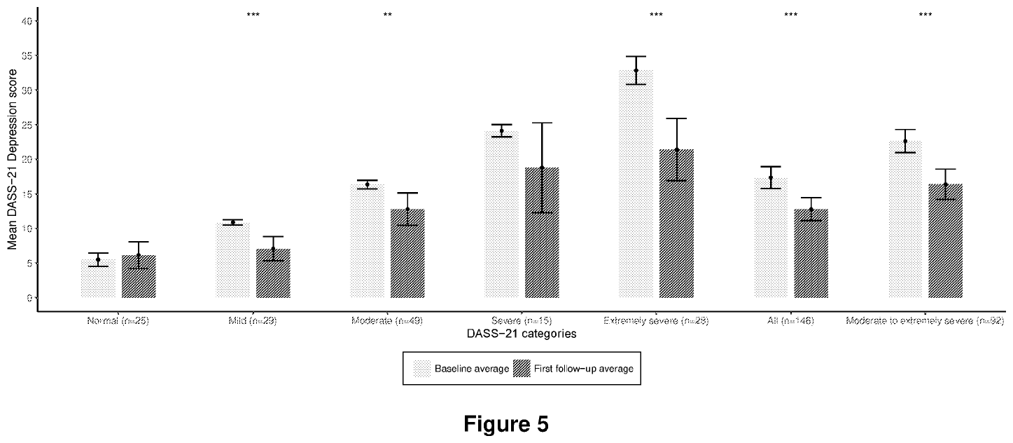

Patients were assessed monthly by the clinical team (doctor and nurse) and a visit schedule protocol was followed for each patient. A full medical history was conducted at the initial visit, including a detailed history of the clinical indication (including duration symptoms), comorbidities mental and cardiac health conditions, previous procedures, medications and therapies tried. Patients were reviewed as per the Schedule of Assessments (FIG. 2). Cannabinoid dose and Adverse events were also recorded at each visit after treatment began and categorised as mild (no product change indicated), moderate (product change indicated), severe (product cessation indicated) or serious (life-threatening/medical emergency).

### Dosing of Medicinal Cannabis Products

The doctors treating patients prescribed orally-based medicinal cannabis standardised products as per approval under the Therapeutic Goods Administration (TGA) through the Special Access Scheme (SAS) Category B and Authorised Prescriber (AP) scheme. The prescribing doctor will decide which product type including THC/CBD ratio and CBD only, based on TGA guidance documents (ie for pain THC/CBD type is generally prescribed) [see https://www.tga.gov.au/medicinal-cannabis-guidance-documents], but also will factor in patient's circumstances such as driving (then CBD only prescribed).

Treatment dosing by the prescribing doctor was in accordance with Emyria (Emerald Clinics) experience, well-tolerated with minimal side effects. Doctors reviewed patients after the 14 day (2 week), see the Schedule of Assessments (FIG. 2). Following the 2-week dosing review where a doctor may increase/decrease the dosage or change product type, patients were then seen regularly (generally monthly) as per the Schedule of Assessments (FIG. 2). Patients taking CBD only products were prescribed either oil or capsules.

### Population Criteria Collected for Evaluation

Population descriptors
   Age
   Sex
   Ethnicity
   Work Status
   Comorbidities
   Family history of diseases
Condition being treated:
   Primary condition being treated
   Any relevant co-morbidities that are being managed that are relevant to the primary condition.
   Prior treatments trialled including medications, surgery and management interventions.
Prior Treatments:
   Concurrent medications
   Surgical procedures
   Other concurrent management interventions (peer support, directly observed therapy)
Comorbidities
   Mental health condition (PTSD, Anxiety, Depression, Other)
   Diabetes
   Cancer
   Heart and circulation problems (including Heart Disease, Pacemaker, Blood Disease)
   Muscle, bone and joint problems
   Respiratory problems (including asthma, lung disease, COPD, sleep apnoea)
   Neurological problems (including stroke, epilepsy, multiple sclerosis, Parkinson's Disease)
   Digestive problems (including IBS, GORD, stomach ulcers, reflux, bowel disease)
   Liver, kidney and pancreas problems (including pancreatitis, kidney disease)
   Thyroid problems (including hyperactive or hypoactive thyroid, Graves' disease)
   Sleep issues (ie trouble falling or staying asleep, restless legs, snoring, terrors, falling asleep without warning)
   Bacterial, fungal, viral or parasitic disease
   Ear, nose, throat, mouth or eye disease
   Skin or connective tissue disease
   Substance abuse
   Wounds or Injuries
   Conditions present at birth
   Other health conditions
Treatment outcome
   Self-reported adherence to cannabis-based therapy expressed as a proportion of all prescribed doses taken
   Self-reported pain, mood (anxiety, depression, stress), sleep and quality of life (QoL) score using clinically validated questionnaires reported monthly for the first year, with regular follow up. See the Schedule of Assessments (FIG. 2).
   Safety Data (recording of Adverse events) at each visit.
   Cannabinoid dose (recording of dose and frequency) at each visit.
   Concomitant medications (recording of medications) at each visit.
   Vital Signs (at each clinic visit)
   Any clinical assessments, laboratory and pathology assessments conducted by GP or specialist as part of their standard treatment of their clinical indication.

### Safety Evaluations

Adverse events (AE) were recorded at each of the visits. AEs were categorised as either: Mild (required no change in dose or product), Moderate (required change in dose or product), Severe (required cessation of product) or Serious (medical emergency).

All AEs reported during treatment were recorded. Assessments included monitoring of any or all of the following parameters: the participant's clinical symptoms, laboratory, pathological, radiological or surgical history, physical examination findings, and/or or findings from blood/urine tests. The nature, time of onset, duration and severity will be documented, together with the Investigator's opinion of the relationship to the Medicinal cannabis product.

Any pre-existing conditions or signs and/or symptoms present in a participant prior to the start of treatment (i.e., before informed consent obtained) were recorded as Medical/Surgical History. In addition, any medical occurrence that is reported after informed consent was obtained, but prior to first starting the medicinal cannabis product, were documented as baseline signs and symptoms.

The screening visit also provided a baseline general health assessment including blood pressure and any pre-existing conditions requiring assessments of kidney and liver function. Participants were monitored for any medications that may interact with medicinal cannabis.

All AEs for patients on CBD only were mild (required no change in dose or product) or moderate (required change in dose or product). Only 36 out of 190 patients had any AEs (19%). For those 36 patients that experienced AEs, 61% of the AEs were mild and 36% were moderate requiring change in dose or product. One of the 36 patients (2.8%) had severe AEs (allergic reactions and constipation) which resulted in cessation of the product. Participants with any AE were monitored regularly and many of the AEs were either single episodes or intermittent and resolved with change of dose or product.

Review of the dose and side effects was conducted by the doctor at 2 weeks; the doctors then advise whether to increase/or decreases doses. Reviews and clinical visits beyond the 2 weeks occurred monthly or more often as required by the patient due to efficacy and side effects.

There were 190 patients with DASS-21 scores in the initial (baseline) visit and follow-up after taking CBD for at least one month with age range 4-95 years (Table 1). 56% of the patients were female and 44% of patients were male.

TABLE 1
Demographics

| Gender | n | Age | Yrs |
|---|---|---|---|
| Female | 106 | ave | 51.5 |
| Male | 84 | med | 52 |
| % Female | 56 | n | 190 |
| % Male | 44 | | |

Lowest 4
Highest 95

Table 2 shows the clinical indications of the patients being treated with cannabidiol.

TABLE 2
Clinical Indications

| Clinical indications | n | % |
|---|---|---|
| Chronic Non-Cancer Pain | 125 | 65.8 |
| Migraine | 3 | 1.6 |
| Insomnia | 8 | 4.2 |
| Anxiety or other mental illness | 5 | 2.6 |
| Post-traumatic Stress Disorder (PTSD) | 7 | 3.7 |
| Epilepsy | 5 | 2.6 |
| Cancer Pain | 2 | 1.1 |
| Others* | 35 | 18.4 |
| Total | 190 | |

*Inflammatory Bowel Disease, neurological spasticity, Parkinson's Disease, autism, sleep disorder, nausea, Chemotherapy Induced Nausea and Vomiting (CINV)

Out of 190 patients, 146 patients had a higher than normal score (mild-extremely severe) in at least one of the categories depression, anxiety and stress using the DASS-21 questionnaire and were analysed for the effect of CBD on depression, anxiety and stress scores post one month. In addition, 79 of the 146 patients (54.1%) also self-reported at least one mental health comorbidity such as anxiety, depression, post-traumatic stress disorder or other mental health condition.

### Dose

Patients were administered between 10 mg and 400 mg cannabidiol per day.

### Results

Table 3 shows the results for 146 patients that had a higher than normal score (mild to extremely severe categories) in at least one of the categories depression/anxiety/stress at the initial visit (baseline) before taking any CBD. Table 3 shows the number of patients with scores in the categories normal, mild, moderate, severe, and extremely severe for depression, anxiety and stress. Mean score results initial mean, with standard deviation are shown at the initial visit before taking CBD and mean with standard deviation results at the one-month follow-up after taking CBD. p-value showing a difference in mean at first follow-up after treatment with CBD and initial visit (baseline).

TABLE 3
Results

| | Depression | | | |
|---|---|---|---|---|
| | Score | Initial Mean | Post 1-month | |
| | Severity | (Std. dev.) | follow-up Mean | |
| DASS-21 Depression Score | Categories | (baseline) | (Std. dev) | p-value |
| Normal (n = 25) | 0-9 | 5.5 (2.3) | 6.2 (4.8) | 0.543 |
| Mild (n = 29) | 10-13 | 10.9 (1.0) | 7.1 (4.6) | 0.000*** |
| Moderate (n = 49) | 14-20 | 16.4 (2.2) | 12.8 (8.1) | 0.002** |
| Severe (n = 15) | 21-27 | 24.1 (1.6) | 18.8 (11.7) | 0.089 |
| Extremely Severe (n = 28) | 28+ | 32.9 (5.2) | 21.4 (11.6) | 0.000*** |
| All (n = 146) | | 17.4 (9.6) | 12.8 (10.0) | 0.000*** |
| Mod to extreme (n = 92) | | 22.7 (8.0) | 16.4 (10.6) | 0.000*** |
| | Anxiety | | | |
| | Score | Initial Mean | Post 1-month | |
| | Severity | (Std. dev.) | follow-up Mean | |
| DASS-21 Anxiety Score | Categories | (baseline) | (Std. dev) | p-value |
| Normal (n = 33) | 0-7 | 3.9 (1.7) | 4.2 (3.7) | 0.667 |
| Mild (n = 13) | 8-9 | 8.0 (0.0) | 7.1 (3.0) | 0.291 |
| Moderate (n = 50) | 10-14 | 12.1 (1.7) | 8.8 (5.1) | 0.000*** |
| Severe (n = 16) | 15-19 | 17.0 (1.0) | 10.9 (7.9) | 0.009** |
| Extremely Severe (n = 34) | 20+ | 26.4 (6.2) | 14.4 (8.1) | 0.000*** |
| All (n = 146) | | 13.7 (8.7) | 9.1 (6.8) | 0.000*** |
| Mod to extreme (n = 100) | | 17.7 (7.5) | 11.0 (7.1) | 0.000*** |
| | Stress | | | |
| | Score | Initial Mean | Post 1-month | |
| | Severity | (Std. dev.) | follow-up Mean | |
| DASS-21 Stress Score | Categories | (baseline) | (Std. dev) | p-value |
| Normal (n = 40) | 0-14 | 10.6 (3.1) | 9.0 (6.0) | 0.100 |
| Mild (n = 27) | 15-18 | 16.9 (1.0) | 14.1 (6.5) | 0.038* |
| Moderate (n = 27) | 19-25 | 22.4 (1.8) | 17.2 (7.7) | 0.002** |
| Severe (n = 34) | 26-33 | 28.9 (1.9) | 22.6 (8.4) | 0.000*** |
| Extremely Severe (n = 18) | 34+ | 38.2 (2.4) | 21.7 (12.2) | 0.000*** |
| All (n = 146) | | 21.6 (9.5) | 16.2 (9.5) | 0.000*** |
| Mod to extreme (n = 79) | | 28.8 (6.2) | 20.5 (9.4) | 0.000*** |

Notes:
Mean days of treatment is 47 (+/−23). *p < 0.05, **p < 0.01, ***p < 0.001

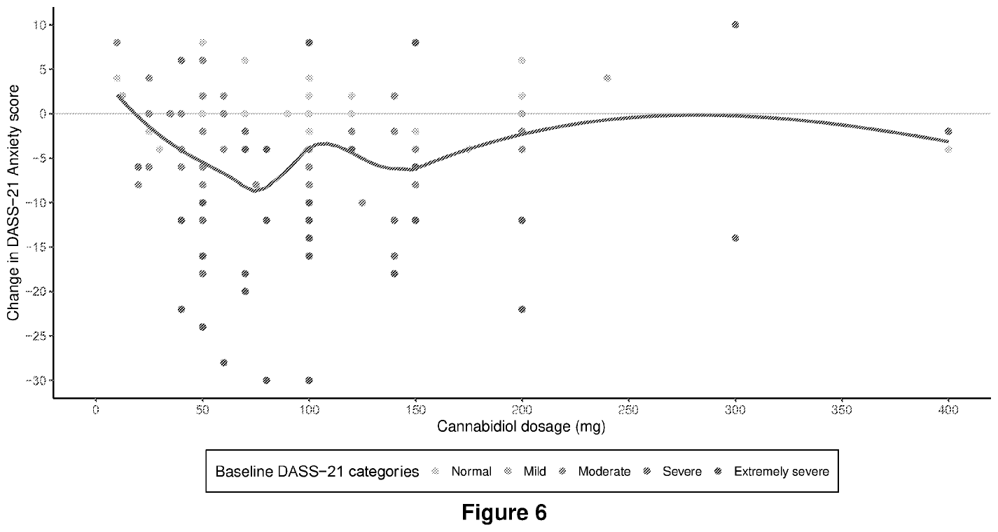

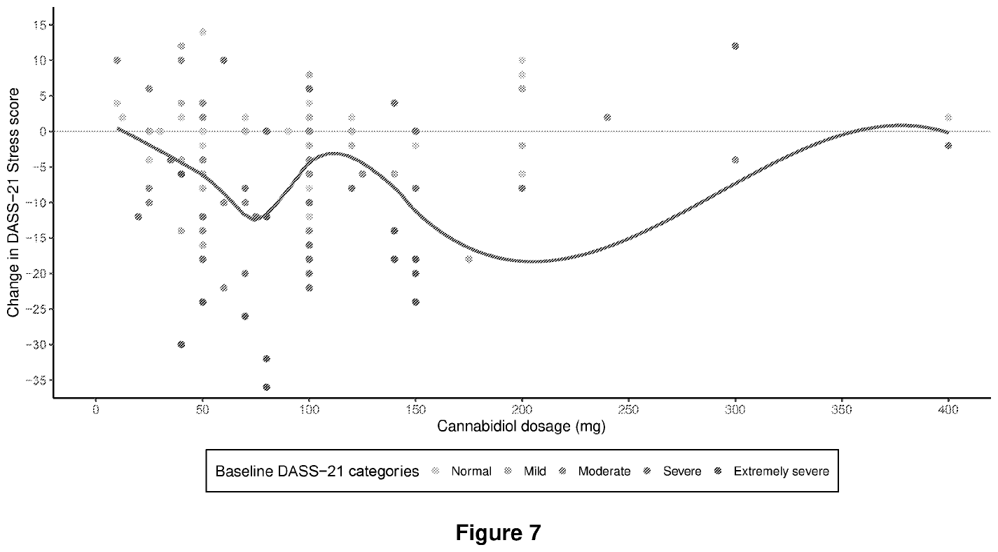

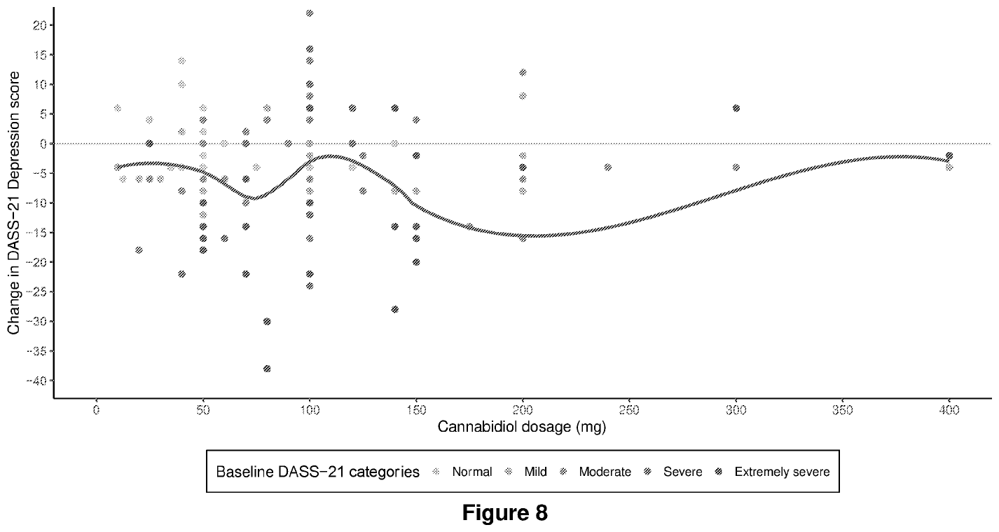

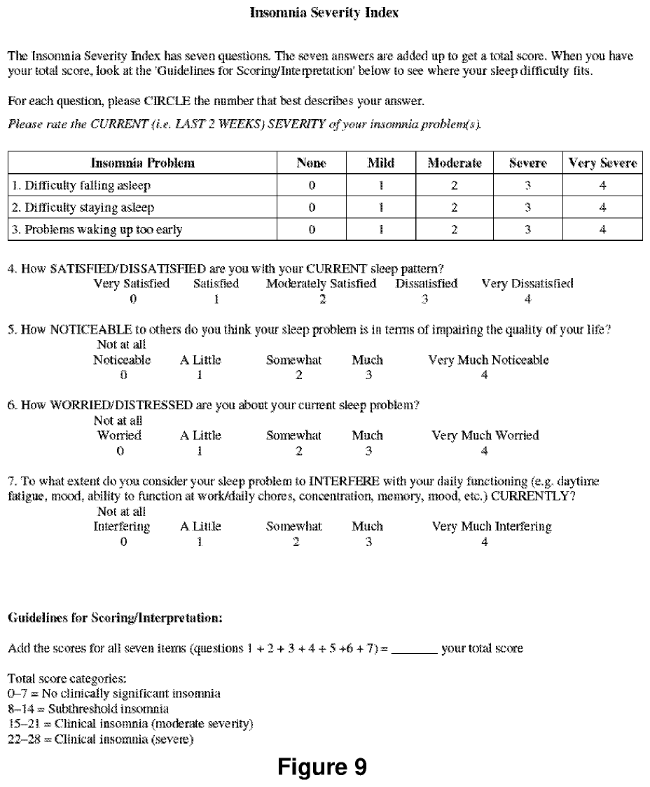

Patients treated with dosage amounts between 50-200 mg per day correlated with a higher value of change indicating less symptoms of anxiety, stress and/or depression at the follow-up visit (see FIGS. 6-8).

### Beyond the First Follow-Up Visit

To explore whether the use of cannabidiol may have a more persistent association with a reduction in psychological distress, the analysis was extended to the first six months of treatment.

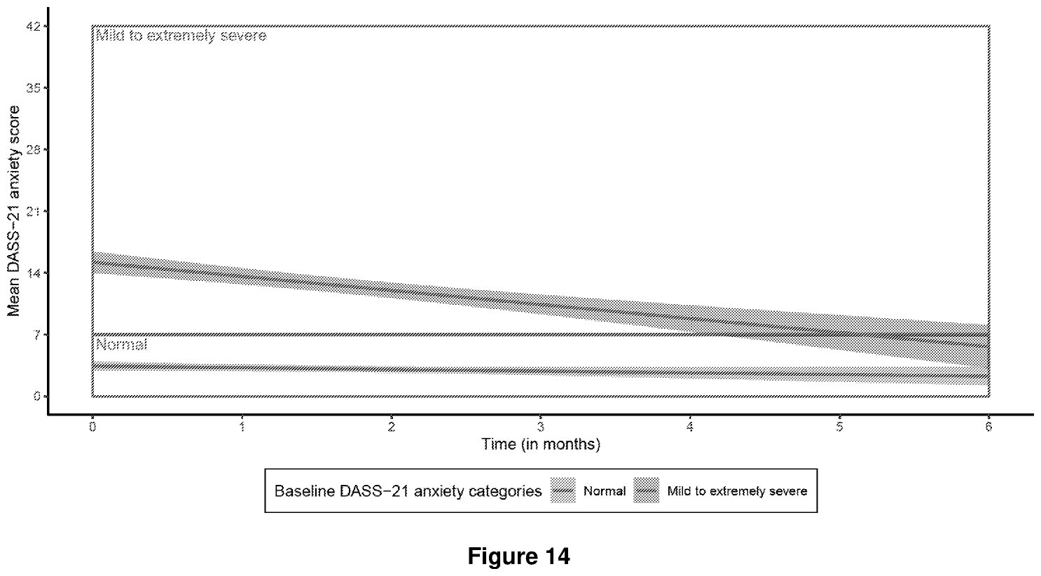

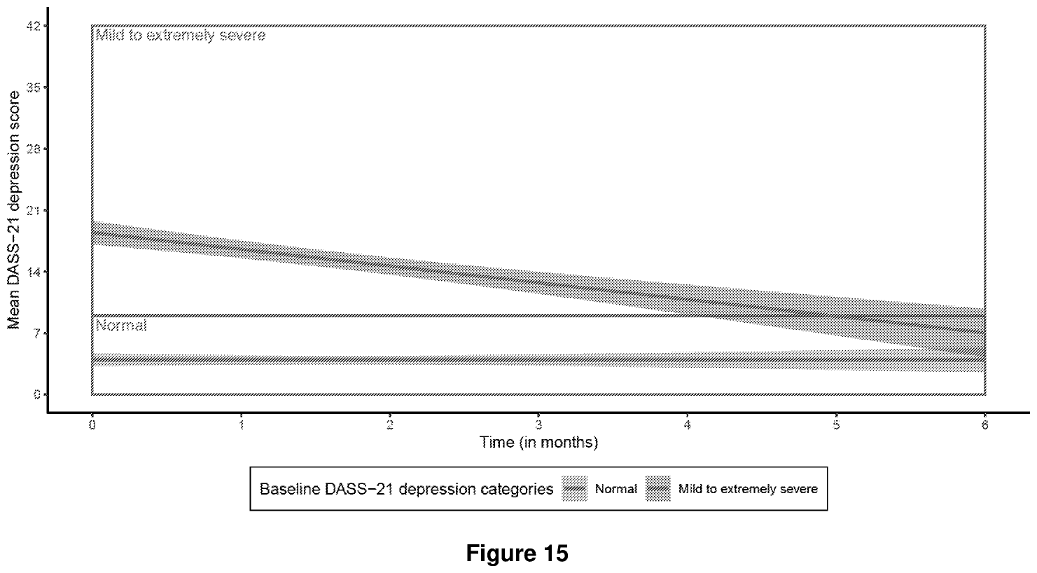

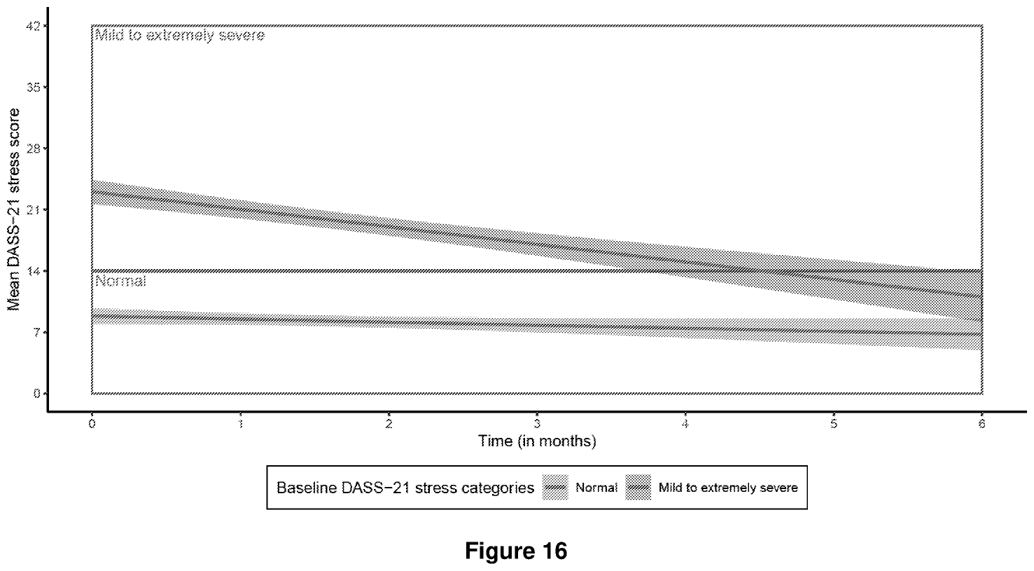

FIGS. 14-16 visualise average DASS-21 anxiety, stress and depression scores over time, where the bands around the solid fitted lines represent the respective 95% confidence intervals. In order to distinguish between patients experiencing above normal symptoms of anxiety, stress and depression and those with symptoms falling in the normal range, the mean score over time is shown by initial assessment at baseline separately. In general, FIGS. 14-16 displays a significant reduction over the 6-month period for patients with subthreshold and clinically significant anxiety, stress and depression scores at baseline.

### Example 2

### Treatment of Insomnia

### Methods

Patients were assessed for inclusion/exclusion using the same screening criteria and population criteria collected for evaluation as in Example 1. Dosing regimens were the same as Example 1.

Tables 4 and 5 document summary statistics for all patients at baseline, in addition to detailed variable descriptions.

Table 4 focuses on key information regarding patients, displaying descriptive data for demographics and mental health conditions (Panel A), primary diagnosis determined at the initial visit (Panel B), and medication used before commencing treatment (Panel C). The mean age of the patients is 51.4 years, 55% are female, and approximately 45% have a mental health condition. Note that mental health conditions are self-identified by the patient at the initial assessment, while primary diagnoses are determined by the treating doctor.

TABLE 4
Summary statistics of patients at baseline (n = 228).

| | | | | | | |
|---|---|---|---|---|---|---|
| | | (Std. | | | | |
| Variable | Mean | Dev.) | Median | Min | Max | Variable definition |

Panel A: Individual characteristics

| | | | | | | |
|---|---|---|---|---|---|---|
| Female | 0.55 | (0.50) | 1 | 0 | 1 | = 1 if patient is female |
| Age | 51.36 | (18.99) | 52 | 4 | 95 | Age (in years) |
| Mental health condition^{a} | 0.45 | (0.50) | 0 | 0 | 1 | = 1 if patient has a mental |
| | | | | | | health condition |
| Anxiety | 0.32 | (0.47) | 0 | 0 | 1 | = 1 if patient has anxiety |
| Depression | 0.28 | (0.45) | 0 | 0 | 1 | = 1 if patient has |
| | | | | | | depression |
| Post-traumatic stress | 0.08 | (0.28) | 0 | 0 | 1 | = 1 if patient has post- |
| disorder | | | | | | traumatic stress disorder |
| | | | | | | (PTSD) |

Panel B: Primary diagnosis

| | | | | | | |
|---|---|---|---|---|---|---|
| Chronic non-cancer pain | 0.54 | (0.50) | 1 | 0 | 1 | = 1 if patient has chronic |
| | | | | | | non-cancer pain (CNCP) |
| Neuropathic pain | 0.19 | (0.40) | 0 | 0 | 1 | = 1 if patient has |
| (peripheral) | | | | | | neuropathic pain |
| | | | | | | (peripheral) relating to |
| | | | | | | CNCP |
| Inflammatory pain | 0.11 | (0.31) | 0 | 0 | 1 | = 1 if patient has |
| | | | | | | inflammatory pain relating |
| | | | | | | to CNCP |
| Neuropathic pain (central) | 0.03 | (0.17) | 0 | 0 | 1 | = 1 if patient has |
| | | | | | | neuropathic pain (central) |
| | | | | | | relating to CNCP |
| Insomnia | 0.06 | (0.23) | 0 | 0 | 1 | = 1 if patient has |
| | | | | | | insomnia |
| Migraine (headache) | 0.06 | (0.23) | 0 | 0 | 1 | = 1 if patient has migraine |
| | | | | | | (headache) |
| Post-traumatic stress | 0.05 | (0.22) | 0 | 0 | 1 | = 1 if patient has post- |
| disorder | | | | | | traumatic stress disorder |
| | | | | | | (PTSD) |
| Epilepsy | 0.03 | (0.17) | 0 | 0 | 1 | = 1 if patient has epilepsy |

Panel C: Pretreatment medication

| | | | | | | |
|---|---|---|---|---|---|---|
| Opioids | 0.43 | (0.50) | 0 | 0 | 1 | = 1 if patient is taking |
| | | | | | | opioids |
| Synthetic opioids | 0.30 | (0.46) | 0 | 0 | 1 | = 1 if patient is taking |
| | | | | | | synthetic opioids |
| Paracetamol | 0.51 | (0.50) | 1 | 0 | 1 | = 1 if patient is taking |
| | | | | | | paracetamol |
| Nonsteroidal anti- | 0.50 | (0.50) | 1 | 0 | 1 | = 1 if patient is taking |
| inflammatory drugs | | | | | | nonsteroidal anti- |
| | | | | | | inflammatory drugs |
| | | | | | | (NSAIDs) |
| Medicinal cannabinoids | 0.02 | (0.13) | 0 | 0 | 1 | = 1 if patient is taking |
| | | | | | | medicinal cannabinoids |
| Benzodiazepines | 0.26 | (0.44) | 0 | 0 | 1 | = 1 if patient is taking |
| | | | | | | benzodiazepines |
| Antidepressants | 0.43 | (0.50) | 0 | 0 | 1 | = 1 if patient is taking |
| | | | | | | antidepressants |
| Anticonvulsants | 0.21 | (0.41) | 0 | 0 | 1 | = 1 if patient is taking |
| | | | | | | anticonvulsants |

Notes:
^{a}Mental health conditions are patient-identified, whereas primary diagnoses displayed in Panel B are determined by the treating doctor. Notice that the specific indications of chronic non-cancer pain and mental health problems do not necessarily add up to the total proportion of cases within the sample (i.e. 0.56 and 0.48), as patients may display multiple indications (in addition to non-specified indications being omitted).

Table 5 presents the main variables with individual ISI scores and categories in Panels A and B, while Panels C and D are dedicated to information regarding prescribed cannabidiol dosages. Note that half of all patients display some level of clinical insomnia at baseline according to 151 categories. The vast majority of cannabidiol dosage prescriptions fall within the 1 mg to 150 mg range.

TABLE 5
Additional summary statistics of patients at baseline (n = 228), focusing on initial DASS-
21 subscale scores and categories.

| | | | | | | |
|---|---|---|---|---|---|---|
| | | (Std. | | | | |
| Variable | Mean | Dev.) | Median | Min | Max | Variable definition |

Panel A: Insomnia Severity Index (ISI) score

| | | | | | | |
|---|---|---|---|---|---|---|
| Insomnia severity | 14.15 | (7.27) | 14 | 0 | 28 | ISI score (increasing from |
| | | | | | | 0 to 28) |

Panel B: Insomnia Severity Index (ISI) categories

| | | | | | | |
|---|---|---|---|---|---|---|
| No clinically significant | 0.22 | (0.41) | 0 | 0 | 1 | = 1 if patient has an |
| insomnia | | | | | | absence of insomnia |
| | | | | | | (ranging from 0 to 7) |
| Subthreshold insomnia | 0.29 | (0.46) | 0 | 0 | 1 | = 1 if patient has |
| | | | | | | subthreshold insomnia |
| | | | | | | (ranging from 8 to 14) |
| Clinical insomnia (moderate | 0.31 | (0.46) | 0 | 0 | 1 | = 1 if patient has moderate |
| severity) | | | | | | insomnia (ranging from 15 |
| | | | | | | to 21) |
| Clinical insomnia (severe) | 0.19 | (0.39) | 0 | 0 | 1 | = 1 if patient has severe |
| | | | | | | insomnia (ranging from 22 |
| | | | | | | to 28) |

Panel C: Prescribed cannabinoid dosage

| | | | | | | |
|---|---|---|---|---|---|---|
| Cannabidiol | 124.35 | (90.42) | 100 | 10 | 600 | Cannabidiol (CBD) dosage |
| | | | | | | (in mg) |

Panel D: CBD dosage ranges

| | | | | | | |
|---|---|---|---|---|---|---|
| CBD 1 mg-50 mg | 0.25 | (0.44) | 0 | 0 | 1 | = 1 if patient is prescribed |
| | | | | | | a CBD dosage between |
| | | | | | | 1mg and 50mg |
| CBD 51 mg-100 mg | 0.40 | (0.49) | 0 | 0 | 1 | = 1 if patient is prescribed |
| | | | | | | a CBD dosage between |
| | | | | | | 51mg and 100mg |
| CBD 101 mg-150 mg | 0.11 | (0.32) | 0 | 0 | 1 | = 1 if patient is prescribed |
| | | | | | | a CBD dosage between |
| | | | | | | 101mg and 150mg |
| CBD ≥150 mg | 0.23 | (0.42) | 0 | 0 | 1 | = 1 if patient is prescribed |
| | | | | | | a CBD dosage greater |
| | | | | | | than 150mg |

To get a better understanding of those who experience different levels of severity of insomnia, Table 6 compares the numbers and proportions of primary diagnoses across the corresponding ISI categories. Most notably, 74.2% of patients with chronic non-cancer pain (CNCP) experience some level of insomnia, with this % rising for those suffering specifically from neuropathic pain (both peripheral and central). Individuals who have been assessed to have insomnia as their primary diagnosis experience either subthreshold or clinically significant insomnia (as measured by the ISI).

TABLE 6
Selection of the most common primary diagnoses across ISI categories at baseline
(n = 228).

| | | | | | Clinical | | | | | |
|---|---|---|---|---|---|---|---|---|---|---|
| | No clinically | | | | insomnia | | Clinical | | | |
| | significant | | Subthreshold | | (moderate | | insomnia | | | |
| | insomnia | | insomnia | | severity) | | (severe) | | Total | |
| Primary diagnosis | n | (%) | n | (%) | n | (%) | n | (%) | n | (%) |
| Chronic non-cancer | 32 | (25.8) | 33 | (26.6) | 38 | (30.6) | 21 | (16.9) | 124 | (100.0) |
| pain | | | | | | | | | | |
| Neuropathic pain | 6 | (13.6) | 11 | (25.0) | 17 | (38.6) | 10 | (22.7) | 44 | (100.0) |
| (peripheral) | | | | | | | | | | |
| Inflammatory pain | 9 | (37.5) | 5 | (20.8) | 4 | (16.7) | 6 | (25.0) | 24 | (100.0) |
| Neuropathic pain | 0 | (0.0) | 4 | (57.1) | 2 | (28.6) | 1 | (14.3) | 7 | (100.0) |
| (central) | | | | | | | | | | |
| Insomnia | 0 | (0.0) | 3 | (23.1) | 5 | (38.5) | 5 | (38.5) | 13 | (100.0) |
| Migraine (headache) | 2 | (15.4) | 6 | (46.2) | 3 | (23.1) | 2 | (15.4) | 13 | (100.0) |
| PTSD | 1 | (9.1) | 0 | (0.0) | 6 | (54.5) | 4 | (36.4) | 11 | (100.0) |
| Epilepsy | 1 | (14.3) | 5 | (71.4) | 1 | (14.3) | 0 | (0.0) | 7 | (100.0) |

### Empirical Results

Table 7 displays the main regression results, predicting short-term therapeutic effects of CBD on ISI scores at the first follow-up visit, average 46.9 days. Column (1) first presents results from a pooled specification with a time specific dummy variable capturing the first follow-up visit (i.e., Visit 1) as the sole explanatory variable. Most notably, the coefficient of interest is negative and statistically significant at the 0.1 percent level, suggesting a decline in insomnia across the treatment period. Notice that the constant term (i.e., the intercept) reflects the average ISI score of 14.145 at baseline.

TABLE 7
Main regression results, predicting short-term change in ISI scores
from baseline to first follow-up (lower values indicate less insomnia).

| Outcome: ISI score | (1) | (2) | (3) |
|---|---|---|---|
| Constant | 14.145*** | | |
| | (0.482) | | |
| Visit 1 | −3.254*** | −3.254*** | 0.959 |
| | (0.393) | (0.393) | (0.646) |
| Visit 1 × subthreshold | | | −3.308*** |
| insomnia | | | (0.901) |
| Visit 1 × clinical insomnia | | | −6.231*** |
| (moderate severity) | | | (0.970) |
| Visit 1 × clinical insomnia | | | −7.122*** |
| (severe) | | | (1.039) |
| Patient fixed effects | | Yes | Yes |
| # of patients | 228 | 228 | 228 |
| # of visits | 2 | 2 | 2 |
| N | 456 | 456 | 456 |
| Mean of outcome (baseline) | 14.145 | 14.145 | 14.145 |

Notes:
The data are from Visit 0 (baseline) and Visit 1 (first follow-up) of patients. Outcome is the short-term change in ISI score between Visit 0 and Visit 1. Standard errors clustered at the patient level aredisplayed in parentheses. *p < 0.05, **p < 0.01, ***p < 0.001.

Column (2) then incorporates patient-fixed effects. Note that when including fixed effects at the person level, the constant term is dropped from the regression, as it is differenced out by the fixed effects estimator—in addition to any other (un)observable time-invariant individual characteristics which may influence ISI scores (such as personality traits or beliefs). As for our coefficient of interest, it continues to emerge as negative and statistically significant. In fact, it remains virtually unchanged from column (1) since the pooled data contains exactly two observations for each patient.

In terms of magnitude, treatment with cannabidiol only of approximately 46.9 days, on average, is suggested to lower an ISI score by −3.254. To place the size of this effect into context, such a reduction would correspond to a substantial 23 percent drop when considering the mean ISI score of 14.145 at baseline (before treatment).

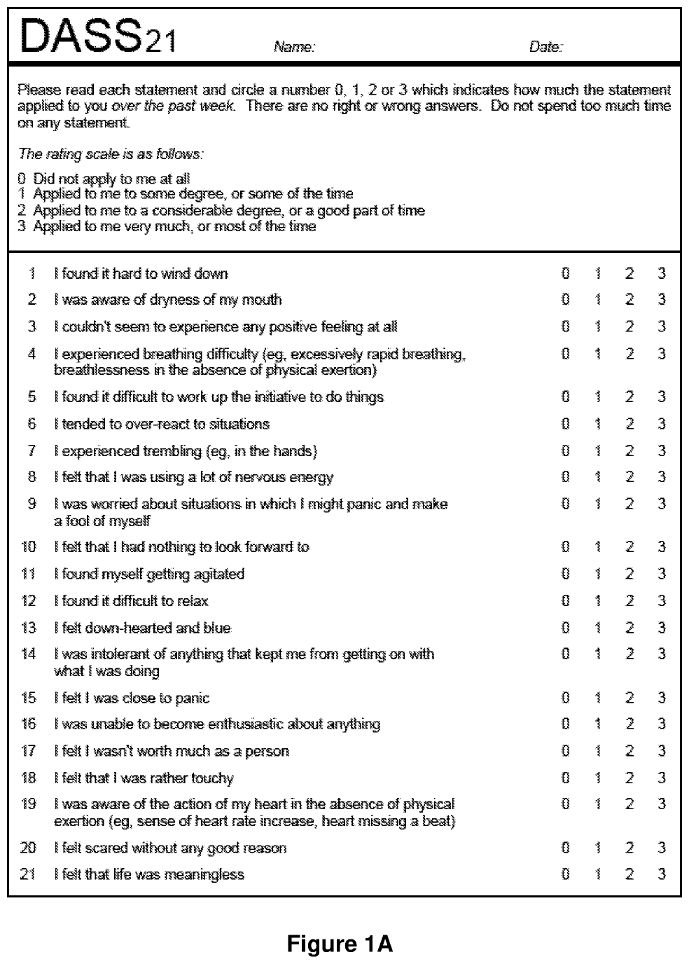

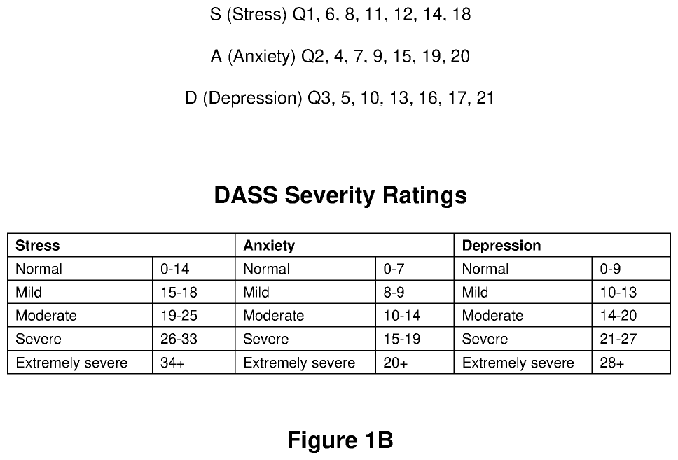

Finally, column (3) introduces interaction terms between the time dummy variable and binary indicators of starting with an ISI score in the ‘subthreshold insomnia’, ‘clinical insomnia (moderate severity)’, or ‘clinical insomnia (severe)’ category at baseline. The reference category here is determined by patients in the ‘no clinically significant insomnia’ category. Consistent with the visuals from FIG. 1, the reduction in insomnia score at the first follow-up is primarily driven by those who start in the ‘subthreshold insomnia’ category or higher at baseline.

Investigating the relationship between cannabidiol treatment and baseline ISI score further, Table 8 presents alternative estimate using initial category-specific subsamples. Patients beginning with an ISI score in the ‘no clinically significant insomnia’ category do not exhibit any meaningful response to cannabidiol treatment, whereas those in the higher categories do.

TABLE 8
Alternative results, estimating ISI scores by initial assessment category.

| | | No | | Clinical | |
|---|---|---|---|---|---|
| | | clinically | | insomnia | Clinical |
| | | significant | Subthreshold | (moderate | insomnia |
| | All | insomnia | insomnia | severity) | (severe) |
| Outcome: ISI score | (1) | (2) | (3) | (4) | (5) |
| Visit 1 | −3.254*** | 0.959 | −2.349*** | −5.271*** | −6.163*** |
| | (0.393) | (0.652) | (0.631) | (0.726) | (0.823) |
| Patient fixed effects | Yes | Yes | Yes | Yes | Yes |
| # of patients | 228 | 49 | 66 | 70 | 43 |
| # of visits | 2 | 2 | 2 | 2 | 2 |
| N | 456 | 98 | 132 | 140 | 86 |
| Mean of outcome | 14.145 | 3.857 | 11.364 | 17.757 | 24.256 |
| (baseline) | | | | | |

Notes:
The data are from Visit 0 (baseline) and Visit 1 (first follow-up) of patients. Outcome is the short-term change in ISI score between Visit 0 and Visit 1. Standard errors clustered at the patient level are displayed in parentheses. *p < 0.05, **p < 0.01, ***p < 0.001.

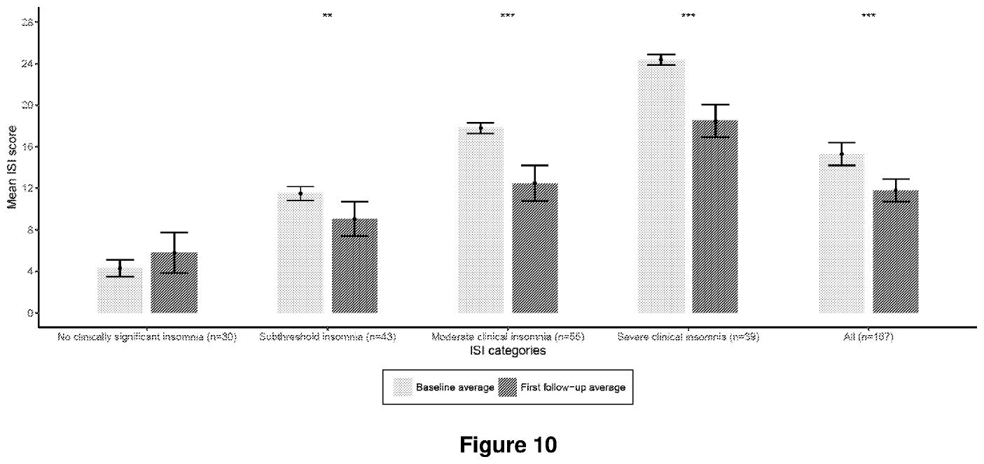

FIG. 10 visualises the short-term changes in ISI scores, comparing mean responses across baseline ISI categories and by visit—initial and first follow-up after being prescribed cannabidiol. Notice that the error bars represent the respective 95 percent confidence intervals, whereas the significance levels are displayed above visit pairs for each category (* p<0.05, ** p<*** p<0.001). It is also worth noting that the mean length of treatment between visits is 46.9 days (median=42 days). Overall, FIG. 10 shows significantly lower mean ISI scores in the first follow-up visit for patients starting with subthreshold insomnia, as well as those with moderate and severe clinical insomnia.

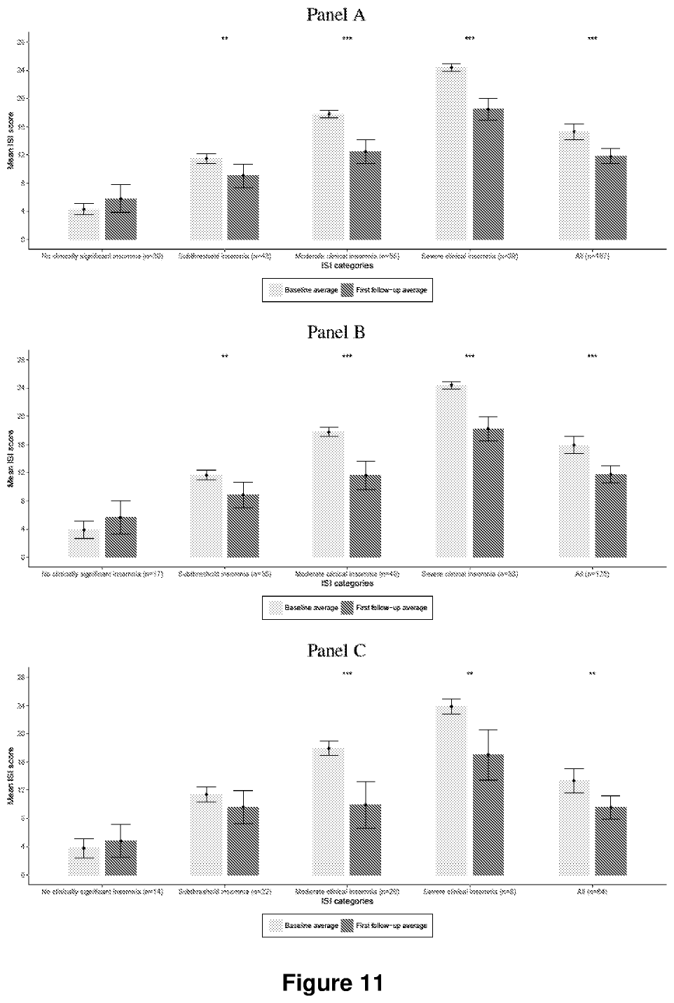

In additional analysis, FIG. 11 plots subsample mean ISI scores across baseline ISI categories and by visit (initial and first follow-up) for patients starting with a (i) DASS-21 anxiety, depression, or stress score above the normal range (Panel A); (ii) DASS-21 anxiety score above the normal range (Panel B); (iii) and DASS-21 anxiety score in either the mild or moderate range (Panel C). In general, FIG. 11 shows remarkably similar differences in ISI scores between visits as shown in FIG. 10.

One of the biggest advantages of the data is that the exact cannabidiol products and dosages prescribed to patients during the treatment period can be examined. In particular, FIG. 11 shows the distribution of CBD dosage across ISI categories at baseline.

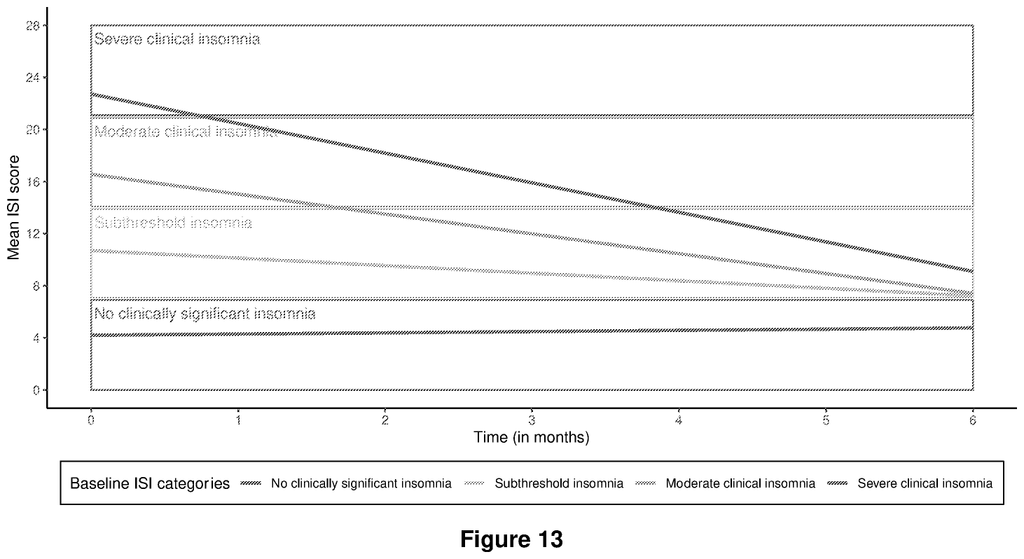

FIG. 13 displays the relationship between cannabidiol dosage and changes in ISI score between visits. Changes in scores are plotted by taking the first difference in values between the first follow-up visit and the initial one. Thus, lower values indicate less insomnia. The colour of each dot represents the ISI category of a patient at their initial visit, with darker shades of red indicating greater severity of insomnia. The solid fitted line is the locally estimated scatterplot smoothing (LOESS) curve with a bandwidth of 0.5.

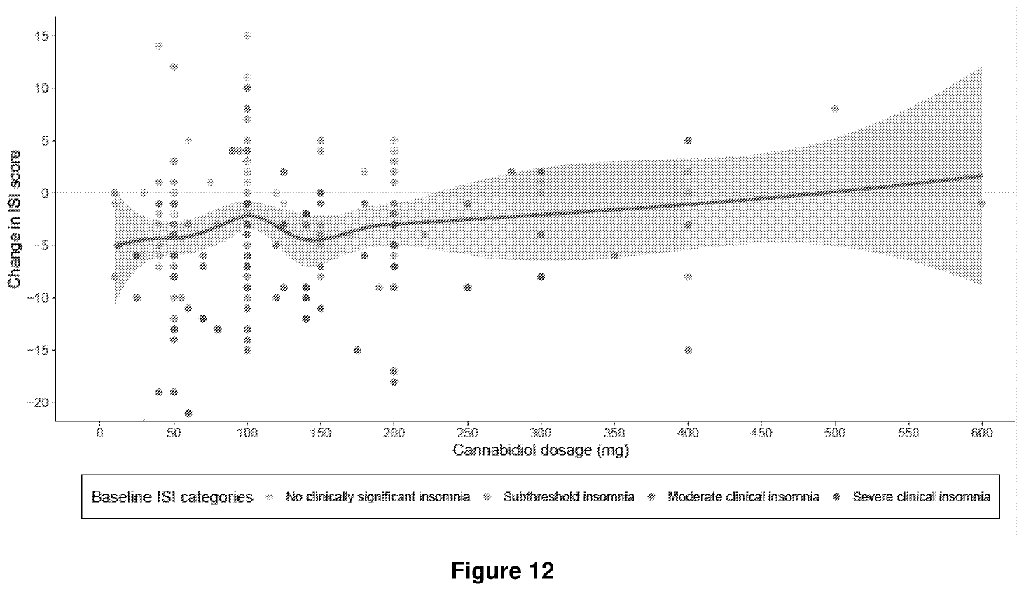

Overall, FIG. 12 shows that patients who are prescribed between approximately 10 mg to 200 mg of cannabidiol report less insomnia at the first follow-up visit.

As a further robustness check (not reported here), whether a particular dosage or dosage range is driving the main findings displayed in Tables 7 and 8 was examined. In particular, the corresponding regressions were re-estimated while incorporating controls for prescribed cannabidiol dosage. The results persist.

### Beyond the First Follow-Up Visit

To explore whether the use of cannabidiol may have a more persistent association with reducing insomnia, the analysis was extended to the first six months of treatment.

FIG. 13 visualises average ISI scores over time, where the bands around the solid fitted lines represent the respective 95% confidence intervals. In order to distinguish between patients experiencing above normal symptoms in insomnia and those with symptoms falling in the normal range, the mean score over time is shown by initial assessment at baseline separately. In general, FIG. 13 displays a significant reduction over the 6-month period for patients with subthreshold and clinically significant insomnia scores at baseline.

Exploring this issue of persistence further, Table 9 presents regression-equation checks. In particular, column (1) replicates the benchmark results showing the change in 151 score between baseline and the first follow-up visit. Columns (2) and columns (3) then re-estimate the results between baseline and the second follow-up visit, and baseline and the third follow-up visit respectively. It can be seen that the decrease in insomnia severity persists, even after 4 and 6 months (i.e., Visits 2 and 3) of commencing CBD only treatment.

TABLE 9
Alternative results, considering further follow-up visits.

| Outcome: ISI score | (1) | (2) | (3) |
|---|---|---|---|
| Visit 1 | −3.254*** | | |
| | (0.393) | | |
| Visit 2 | | −4.141*** | |
| | | (0.581) | |
| Visit 3 | | | −4.313*** |
| | | | (0.854) |
| Patient fixed effects | Yes | Yes | Yes |
| # of patients | 228 | 228 | 228 |
| # of visits | 2 | 2 | 2 |
| N | 456 | 349 | 292 |
| Mean of outcome (baseline) | 14.145 | 14.145 | 14.145 |

Notes:
The data are from Visit 0 (baseline), Visit 1 (first follow-up), Visit 2 (second follow-up), and Visit 3 (third follow-up) of patients. Outcome is (in columns): (1) the short-term change in ISI score between Visit 0 and Visit 1, (2) the short-term change in ISI score between Visit 0 and Visit 2, and (3) the short-term change in ISI score between Visit 0 and Visit 3. Standard errors clustered at the patient level are displayed in parentheses. *p < 0.05, **p < 0.01, ***p < 0.001.

Notice that while the core results focus on treatment effects of CBD only treatment on psychological distress for an average period of 47 days, the findings are also suggestive of clinically meaningful declines in survey scores across shorter time horizons as well. For a mean treatment period of 7 days, the corresponding declines in depression, anxiety, stress, and insomnia scores are −17.5, −10.5, −16.0, and −7.2 respectively. As for an average treatment window of slightly under 13 days, the associated decreases in depression, anxiety, stress, and insomnia scores are −11.2, −6.0, −9.0, and −5.0 respectively. Finally, for a mean treatment horizon of 22 days, the respective falls in depression, anxiety, stress, and insomnia scores are −3.9, −5.7, −5.8, and −4.1.

1. A method of treating one or more symptoms of psychological distress in a subject comprising: administering a dose of between 50 mg/day and 200 mg/day cannabidiol for at least one week, wherein the subject has symptoms of at least one of anxiety, stress and/or depression when assessed by a validated scale or biomarker for identifying symptoms of anxiety, stress and/or depression.

2. The method of claim 1 wherein the subject also has insomnia as assessed by a validated scale or biomarker for identifying symptoms of insomnia.

3. The method of claim 1 wherein the subject has symptoms of two or more of anxiety, stress and/or depression.

4. The method of claim 1 wherein the method:
i. reduces the subject's score on the validated scale for anxiety or reduces the anxiety biomarker;
ii. reduces the subject's score on the validated scale for stress or reduces the stress biomarker; and/or
iii. reduces the subject's score on the validated scale for depression or the depression biomarker.

5. The method of claim 4 wherein the method reduces the subject's score on the validated scale for insomnia or reduces the insomnia biomarker.

6. A composition comprising between 50 mg and 200 mg cannabidiol for the treatment of psychological distress in a subject wherein the subject has symptoms of anxiety, stress and/or depression when assessed by a validated scale or biomarker for identifying symptoms of anxiety, stress and/or depression and wherein the total daily dose of cannabidiol is between 50 mg/day and 200 mg/day.

7. The composition of claim 6 wherein the composition is administered orally, by nasal or pulmonary administration, or administered via the oral mucosa.

8.-9. (canceled)

10. A kit comprising between 50 mg and 200 mg cannabidiol for the treatment [or preventing] of psychological distress in a subject wherein the subject has symptoms of anxiety, stress and/or depression when assessed by a validated scale or biomarker for identifying symptoms of anxiety, stress and/or depression and wherein the total daily dose of cannabidiol is between 50 mg/day and 200 mg/day and wherein the composition is administered for at least one week.

11. The composition according to claim 6, use according to claim 8 or 9 or kit according to claim 10 wherein the subject also has insomnia as assessed by a validated scale or biomarker for identifying symptoms of insomnia.

12. The composition according to claim 6 wherein the subject has symptoms of two or more of anxiety, stress and/or depression.
